(19) United States
(12) Patent Application Publication
(10) Pub. No.: US 2023/0076766 A1
(43) Pub. Date: Mar. 9, 2023

(54) METHOD FOR TREATMENT OF ROSACEA IN PATIENTS AGED 35-64 YEARS

(71) Applicant: Sol-Gel Technologies Ltd., Ness Ziona (IL)

(72) Inventors: Ofer TOLEDANO, Kfar Saba (IL); Ofra LEVY-HACHAM, Ness Ziona (IL); Ori NOV, Tarum (IL); Vered RAM, Rehovot (IL)

(21) Appl. No.: 17/892,620

(22) Filed: Aug. 22, 2022

Related U.S. Application Data
(60) Provisional application No. 63/235,589, filed on Aug. 20, 2021.

(51) Int. Cl. A61K 8/38; A61K 8/67; A61P 17/00; A61K 9/48

(52) U.S. Cl. CPC A61K 8/38; A61K 8/67; A61P 17/00; A61K 9/4808

(57) ABSTRACT

A regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, at least about 4 weeks, at least about 8 weeks, or at least about 12 weeks, to achieve, in a group of such subjects, a success rate of at least about 12%, at least about 27%, at least about 38%, or at least about 42%, respectively, and wherein the success rate is defined as the number of subjects achieving clear or almost clear skin on the investor global assessment (IGA) scale after treatment with the pharmaceutical composition.

### CROSS-REFERENCE TO RELATED APPLICATIONS

This application claims priority under 35 U.S.C. § 119(e) from U.S. Provisional Application No. 63/235,589, filed Aug. 20, 2021, which is incorporated in its entirety as if fully set forth herein.

### TECHNICAL FIELD

This application relates to methods for the therapeutic treatment of symptoms and considerations associated with skin conditions and afflictions, such as rosacea, in patients aged 35-64 years, including topically applying to the skin of a subject in need of said treatment a pharmaceutical composition comprising benzoyl peroxide.

### BACKGROUND

Rosacea is a chronic disease of inflammatory dermatitis that mainly affects the median part of the face and the eyelids of certain adults. It is characterized by telangiectatic erythema, dryness of the skin, papules and pustules. Conventionally, rosacea develops in adults from the ages of 30 to 50. Rosacea is usually a chronic condition, and its prevalence and severity can increase as populations advance in age. Thus, treatment of rosacea in patients who are aged 35-64 years can be more challenging compared to younger adult patients (e.g., patients aged 18-34). Women are more frequently affected by rosacea, although the condition is generally more severe in men. Rosacea is a primitively vascular condition whose inflammatory stage lacks the cysts and comedones characteristic of common acne.

Factors that have been described as possibly contributing towards the development of rosacea include, for example: the presence of parasites such as the Demodex folliculorum; the presence of bacteria such as Helicobacter pylori (a bacterium associated with gastrointestinal disorders); hormonal factors (such as endocrine factors); climatic and immunological factors; and so forth.

Rosacea develops in four stages over several years, in spasms aggravated by variations in temperature, alcohol, spices, exposure to sunlight and stress. The various stages of the disease are:

Stage 1 (stage of erythema episodes): the patients have erythrosis spasms due to the sudden dilation of the arterioles of the face, which then take on a congestive, red appearance. These spasms are caused by emotions, meals and temperature changes.

Stage 2 (stage of couperosis, i.e., of permanent erythema with telangiectasia): certain patients also have oedema on the cheeks and the forehead.

Stage 3 (inflammatory stage, papulopustular rosacea): patients exhibit appearance of inflammatory papules and pustules, but without affecting the sebaceous follicles, and thus, does not include cysts and comedones.

Stage 4 (rhinophyma stage): this late phase essentially affects men. The patients have a bumpy, voluminous red nose with sebaceous hyperplasia and fibrous reordering of the connective tissue.

Erythema is a symptom of rosacea, and includes an abnormal and/or superficial reddening and inflammation of the skin, usually localized or patchy, caused by the congestion and dilation of blood capillaries.

Typical treatment of rosacea includes oral or topical administration of antibiotics such as tetracyclines, salicylic acid, anti-fungal agents, steroids, metronidazole (an anti-bacterial agent) and isotretinoin, or treatment with anti-infectious agents such as azelaic acid.

### SUMMARY

An exemplary embodiment of this application is a regimen for the therapeutic treatment of rosacea in a subject aged 35-64 years, the regimen comprising topically applying to the skin of a subject in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, to achieve, in a group of such subjects, a success rate of at least about 12%, wherein the success rate is defined as the number of subjects achieving clear or almost clear skin on the investor global assessment (IGA) scale after treatment with the pharmaceutical composition.

In another exemplary embodiment, the success rate after about 2 weeks of treatment is at least about 13%.

In another exemplary embodiment, the success rate of said regimen after treatment for about 2 weeks of treatment is at least about 8% greater than a success rate achieved by a vehicle control (e.g., a vehicle cream).

Another exemplary embodiment of this application is a regimen for the therapeutic treatment of rosacea in a subject aged 35-64 years, the regimen comprising topically applying to the skin of a subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks, to achieve, in a group of such subjects, a success rate of at least about 27%, wherein the success rate is defined as the number of subjects achieving clear or almost clear skin on the investor global assessment (IGA) scale after treatment with the pharmaceutical composition.

In another exemplary embodiment, the success rate after about 4 weeks of treatment is at least about 30%.

In another exemplary embodiment, the success rate of said regimen after treatment for about 4 weeks is at least about 17% greater than a success rate achieved by a vehicle control (e.g., a vehicle cream).

Another exemplary embodiment of this application is a regimen for the therapeutic treatment of rosacea in a subject aged 35-64 years, the regimen comprising topically applying to the skin of the subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks, to achieve, in a group of such subjects, a success rate of at least about 38%, wherein the success rate is defined as the number of subjects achieving clear or almost clear skin on the investor global assessment (IGA) scale after treatment with the pharmaceutical composition.

In another exemplary embodiment, the success rate after about 8 weeks of treatment is at least about 42%.

In another exemplary embodiment, the success rate of said regimen after about 8 weeks of treatment is at least about 17% greater than a success rate achieved by the vehicle control (e.g., a vehicle cream).

Another exemplary embodiment is a regimen for the therapeutic treatment of rosacea in a subject aged 35-64 years, the regimen comprising topically applying to the skin of the subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks, to achieve, in a group of such subjects, a success rate of at least about 42%, wherein the success rate is defined as the number of subjects achieving clear or almost clear skin on the investor global assessment (IGA) scale after treatment with the pharmaceutical composition.

In another exemplary embodiment, the success rate after treatment for about 12 weeks is at least about 47%.

In another exemplary embodiment, the success rate of said regimen after about 12 weeks of treatment, is at least about 22% greater than the success rate achieved by the vehicle control (e.g., a vehicle cream).

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, to achieve, in a group of such subjects, a mean percentage decrease, from baseline, in the number of inflammatory lesions of at least about 38%.

In another exemplary embodiment, the mean percentage decrease is at least about 42%. Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks, to achieve, in a group of such subjects, a mean percentage decrease, from baseline, in the number of inflammatory lesions of at least about 52%.

In another exemplary embodiment, the mean percentage decrease is at least about 58%. Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks to achieve, in a group of such subjects, a mean percentage decrease, from baseline, in the number of inflammatory lesions of at least about 61%.

In another exemplary embodiment, the mean percentage decrease is at least about 68%. Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks, to achieve, in a group of such subjects, a mean percentage decrease, from baseline, in the number of inflammatory lesions of at least about 62%.

In another exemplary embodiment, the mean percentage decrease is at least about 69%. Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, to achieve, in a group of such subjects, a mean percentage decrease, from baseline, in the number of inflammatory lesions of at least about 42% compared to a mean percentage decrease, from baseline, in the inflammatory lesions of at least about 23% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks, to achieve, in a group of such subjects, a mean percentage decrease, from baseline, in the number of inflammatory lesions of at least about 58% compared to a mean percentage decrease, from baseline, in the inflammatory lesions of at least about 36% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks, to achieve, in a group of such subjects, a mean percentage decrease, from baseline, in the number of inflammatory lesions of at least about 68% compared to a mean percentage decrease, from baseline, in the inflammatory lesions of at least about 42% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks, to achieve, in a group of such subjects, a mean percentage decrease, from baseline, in the number of inflammatory lesions of at least about 69% compared to a mean percentage decrease, from baseline, in the inflammatory lesions of at least about 41% after treatment with vehicle control.

In other exemplary embodiments, the benzoyl peroxide is the sole active ingredient administered to the subject during the duration of the regimen.

In other exemplary embodiments, the pharmaceutical composition comprises about 2.5% w/w to about 10% w/w of benzoyl peroxide, preferably about 3% to about 9%, about 4% to about 8%, and more preferably about 5% w/w of benzoyl peroxide.

In other exemplary embodiments, the benzoyl peroxide is in a form selected from the group consisting of solid, solution, or suspension.

In other exemplary embodiments, the regimen is a first line therapy for the treatment of rosacea. In some exemplary embodiments, the regimen is a first line therapy for the treatment of rosacea in a subject aged 35-64 years.

In other exemplary embodiments, the rosacea is any of erythematotelangiectatic rosacea, papulopustular rosacea, phymatous rosacea, ocular rosacea, and combinations thereof.

In other exemplary embodiments, the rosacea is moderate to severe rosacea, preferably severe rosacea.

In other exemplary embodiments, the pharmaceutical composition is a cream or an emulsion.

In other exemplary embodiments, the pharmaceutical composition is an extended release formulation.

In other exemplary embodiments, the benzoyl peroxide is present in an inert system that provides a barrier between the benzoyl peroxide and its surrounding environment.

In other exemplary embodiments, the benzoyl peroxide is released in predetermined amounts over a specified period of time

In other exemplary embodiments, the extended-release effect is obtained by encapsulation, microencapsulation, microspheres, a coating, or a combination thereof, and the like.

In other exemplary embodiments, the benzoyl peroxide is encapsulated or micro encapsulated.

In other exemplary embodiments, the benzoyl peroxide is included in a microsphere or a coating.

In other exemplary embodiments, the regimen comprises topically applying the pharmaceutical composition to the skin of a subject aged 40-64 years, 45-64 years, 50-64 years, 55-64 years, or 60-64 years in need of said treatment

Another exemplary embodiment is a pharmaceutical composition for use in the treatment of rosacea (e.g., severe rosacea) in a subject aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, and said pharmaceutical composition is applied (or capable of being applied) once daily for a period of at least about 2 weeks, at least about 4 weeks, at least about 8 weeks or at least about 12 weeks, to achieve, in a group of such subjects, a success rate of at least about 12%, at least about 27%, at least about 38%, or at least about 42%, respectively, wherein the success rate is defined as the number of subjects achieving clear or almost clear skin on the investor global assessment (IGA) scale after treatment with the pharmaceutical composition.

In other exemplary embodiments, the pharmaceutical composition is applied (or capable of being applied) once daily for a period of at least about 2 weeks, at least about 4 weeks, at least about 8 weeks or at least about 12 weeks, to achieve, in a group of such subjects, a success rate of at least about 13%, at least about 30%, at least about 42%, or at least about 47%, respectively.

In other exemplary embodiments, the success rate of said regimen after at least about 2 weeks, at least about 4 weeks, at least about 8 weeks or at least about 12 weeks, is at least about 8% greater, at least about 17% greater, at least about 17% greater, or at least about 22% greater, respectively, than a success rate achieved after treatment for at least about 2 weeks, at least about 4 weeks, at least about 8 weeks or at least about 12 weeks, respectively, with a vehicle control.

In other exemplary embodiments, the success rate of said regimen, for subjects aged 35-64 years and after treatment for at least about 2 weeks, at least about 4 weeks, at least about 8 weeks, or at least about 12 weeks with the pharmaceutical composition, is at least about 7% greater than a success rate achieved after treatment of subjects aged 18-35 years for at least about 2 weeks, at least about 4 weeks, at least about 8 weeks, or at least about 12 weeks, respectively, with the pharmaceutical composition.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 2 weeks, at least about 4 weeks, at least about 8 weeks, or at least about 12 weeks, to achieve, in a group of such subjects, a mean percentage decrease, from baseline, in the number of inflammatory lesions of at least about 38%, at least about 52%, at least about 61%, or at least about 62%, respectively.

In other exemplary embodiments, said pharmaceutical composition is applied (or capable of being applied) once daily for a period of at least about 2 weeks, at least about 4 weeks, at least about 8 weeks, or at least about 12 weeks, to achieve, in a group of such subjects, a mean percentage decrease, from baseline, in the number of inflammatory lesions of at least about 42%, at least about 58%, at least about 68%, or at least about 69%, respectively.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 2 weeks, at least about 4 weeks, at least about 8 weeks, or at least about 12 weeks, to achieve, in a group of such subjects, a mean percentage decrease, from baseline, in the number of inflammatory lesions of about 42%, about 58%, about 68%, or about 69%, respectively, compared to a mean percentage decrease, from baseline, in the inflammatory lesions of about 23%, about 36%, about 42%, or about 41%, respectively, after treatment with vehicle control.

In other exemplary embodiments, the pharmaceutical composition comprises about 2.5% w/w to about 10% w/w of benzoyl peroxide, preferably about 3% to about 9%, about 4% to about 8%, and more preferably about 5% w/w of benzoyl peroxide.

In other exemplary embodiments, the benzoyl peroxide is in a form selected from the group consisting of solid, solution, or suspension.

In other exemplary embodiments, the pharmaceutical composition is applied (or capable of being applied) as a first line therapy for the treatment of rosacea. In some exemplary embodiments, the pharmaceutical composition is applied (or capable of being applied) as a first line therapy for the treatment of rosacea in a subject aged 35-64 years.

In other exemplary embodiments, the rosacea is any of erythematotelangiectatic rosacea, papulopustular rosacea, phymatous rosacea, ocular rosacea, and combinations thereof.

In other exemplary embodiments, the rosacea is moderate to severe rosacea, preferably severe rosacea.

In other exemplary embodiments, the pharmaceutical composition is a cream or an emulsion.

In other exemplary embodiments, the pharmaceutical composition is an extended release formulation.

In other exemplary embodiments, the benzoyl peroxide is present in an inert system that provides a barrier between the benzoyl peroxide and its surrounding environment.

In other exemplary embodiments, the benzoyl peroxide is released (or capable of release) in predetermined amounts over a specified period of time

In other exemplary embodiments, the extended-release effect is obtained by encapsulation, microencapsulation, microspheres, a coating, or a combination thereof, and the like.

In other exemplary embodiments, the benzoyl peroxide is encapsulated or micro encapsulated.

In other exemplary embodiments, the benzoyl peroxide is included in a microsphere or a coating.

In other exemplary embodiments, the pharmaceutical composition is applied (or capable of being applied) to the skin of a subject aged 40-64 years, 45-64 years, 50-64 years, 55-64 years, or 60-64 years, in need of said treatment. In other exemplary embodiments, the regimen comprises topically applying the pharmaceutical composition to the skin of a subject aged 35-60 years, 35-55 years, 35-50 years, 35-45 years, or 35-40 years in need of said treatment.

Details of other exemplary embodiments of the present disclosure will be included in the following detailed description and the accompanying drawings. It is appreciated that certain features of the exemplary embodiments described in this application, which are, for clarity, described in the context of separate embodiments, can also be provided in combination in a single embodiment.

### BRIEF DESCRIPTION OF THE DRAWINGS

In order to understand the disclosure and to see how it can be carried out in practice, embodiments will now be described, by way of non-limiting examples only, with reference to the accompanying drawings, in which:

FIG. 1 is a bar graph of the results of Table 1, showing the success rate of treatment in patients aged 35-64 years based on the investigator global assessment (IGA) scale.

FIG. 2 is a bar graph of the results of Table 2, showing the mean percentage decrease in the number of inflammatory lesions in patients aged 35-64 years.

### DETAILED DESCRIPTION

Multiple studies have been directed to the treatment of rosacea using a pharmaceutical or dermatological active agent such as metronidazole, azelaic acid, sulfacetamide, brimonidine, ivermectin, permethrin, and with doxycycline, which are identified as the only FDA-approved treatments for rosacea (Oge et al., “Rosacea: Diagnosis and Treatment,” American Family Physician, v. 92(3), pp. 187-198 (2015); Gul et al., “A case of granulomatous rosacea successfully treated with pimecrolimus cream,” J. Derm. Treatment, 19, 313-315 (2008)).

Benzoyl peroxide (BPO) is generally identified as an anti-acne agent, used alone (U.S. Pat. No. 9,439,857; Wester et al., “Controlled release of benzoyl peroxide from a porous microsphere polymeric system can reduce topical irritancy,” J. Am. Acad. Derma. 24, 720-726 (1991); Sawleshwarkar, “Multicenter study to evaluate efficacy and irritation potential of benzoyl peroxide 4% cream in hydrophase base (Brevoxyl) in acne vulgaris,” Ind. J. Derm. Vener. Lepro., 69(1), 19-22 (2003)) or in combination with a primary active such as avermectin (U.S. 2011/0052515).

One such study includes a therapeutic regimen involving treatment of acne rosacea in a group of patients in need of such treatment with 5% BPO-acetone gel for four weeks, followed by treatment of the same group of patients with 10% BPO-acetone gel for an additional four weeks. (Montes et al., “Topical Treatment of Acne Rosacea with Benzoyl Peroxide Acetone Gel,” Therapeutics for the Clinician: New Reports on Treatment Modalities of Possible Interest to Patient-Caring Physicians, 32, 185-190 (1983)). The Montes study showed a significantly better response during the five to eight weeks of treatment with 10% BPO-acetone gel compared to the first four weeks of treatment with 5% BPO-acetone gel. Moreover, although Montes 1983 claims success in the treatment of rosacea using a BPO-acetone gel, 25% of the patients in the study showed no improvement and 40% of the patients developed an irritation. Additionally, this study required increasing the amount of BPO administered to the patients from 5% to 10% after week four. The results of the Montes 1983 study make it clear that BPO would not be suitable for regular use in the treatment of rosacea, especially as a first line treatment of rosacea.

Recommendations from the global ROSacea COnsensus (ROSCO) panel, an international panel of dermatologists and ophthalmologists who developed consensus recommendation for rosacea treatment, include using doxycycline- and/or metronidazole-based formulations, alone or in combination with other tetracyclines, as a first-line treatment for rosacea. (Schaller et al., “Rosacea treatment update: Recommendations from the global ROSacea consensus (ROSCO) panel, 13 pages (2016)). The ROSCO panel does not identify the use of BPO in a first-line treatment of rosacea. Furthermore, BPO is notably absent from many other recognized treatment guidelines for the treatment of rosacea. (Treatment Guidelines for Rosacea, United Kingdom National Health Service, 3 pages (2019); RIVERO et al., “An update on the treatment of rosacea,” Australian Prescriber, 41(1), 20-24 (2018)). More recently, standard management options for rosacea were updated in 2019 by the National Rosacea Expert Committee. (Thiboutot et al., Standard management options for rosacea: The 2019 update by the National Rosacea Society Expert Committee, J. Am. Acad. Dermatol., 82, 1501-10 (2020)). The Committee and related references include various treatment options for diagnostic features, major features and ocular rosacea. None of the options identified include BP.

Other studies show that, when used in the treatment of rosacea, BPO is generally combined with a primary active agent such as clindamycin (Breneman et al., “Double-blind, randomized, vehicle-controlled clinical trial of once-daily benzoyl peroxide/clindamycin topical gel in the treatment of patients with severe rosacea,” Int. J. Derm., 43, 381-387 (2004); Gold et al., “Use of Benzoyl Peroxide/Clindamycin gel in the once daily treatment of moderate rosacea,” J. Amer. Acad. Dermat., 52(3), sup., P25 (2004); Leyden et al., “Blind photographic review for a double blind, multicenter, placebo-controlled study comparing Benzoyl Peroxide/Clindamycin and placebo for the treatment of rosacea,” J. Amer. Acad. Dermat., 52(3), sup., P14 (2004); Goldgar et al., “Treatment Options for Acne Rosacea,” J. Amer. Fam. Physician, 80(5), 461-468 (2009)). Engin et al. describes various conventional and novel treatment modalities in rosacea. (Engin et al., Conventional and Novel Treatment Modalities in Rosacea, Clin. Cosm. Investig. Dermatology, 13, 179-186 (2020)). However, Engin et al. do not recommend using BPO as a sole active agent, let alone as a first-line agent, for the treatment of rosacea. Engin et al. merely propose using BPO in combination with other active agents, such as erythromycin, clarithromycin, and the like.

BPO is generally identified as only a possible second-line treatment of rosacea following the use of another, different active. (Oge 2015, Table 5; Goldgar 2009, “Key Recommendations for Practice”). Goldgar 2009, in particular, recommends the use of BPO only as a tertiary therapy for the treatment of rosacea.

When BPO was used as the sole active agent for the treatment of rosacea, lesions were found to be unresponsive. (Gul 2008).

These previous rosacea treatments with BPO alone or in combination with other agents, have been shown to have several drawbacks such as irritation and intolerance phenomena, especially when they are administered for a prolonged period. (Crawford et al., “Rosacea: I. Etiology, pathogenesis, and subtype classification,” J. Am. Acad. Dermatol., 51, 327-341 (2004)). These treatments are only suppressive and not curative, acting especially on the pustulous spasms occurring during the inflammatory stage.

Human maximization tests and patch testing suggest that the sensitization rate of BPO is quite high. (Morelli et al., “Contact dermatitis due to benzoyl peroxide” Contact Dermatitis (Short Communication), 20, 238-39 (1989); see also Morillas et al., “Is benzoyl peroxide an irritant or sensitizer?” Contact Dermatitis, 16:232-33 (1987)). BPO is a recognized cause of allergic contact dermatitis (ACD), and there are multiple cases of patients exhibiting acute and progressive facial erythema and edema, and scaling, after using BPO-containing compositions for treating various ailments. (Felton et al., Benzoyl Peroxide in Topical Acne Preparations: An Underreported Contact Allergen? Dermatitis, 24(3), 146-47 (2013)). In other reported cases of allergic contact dermatitis to BPO, the facial skin of a first patient was severely edematous, red and burning overnight after a single application of a 5% BPO preparation, and a second patient noted marked facial erythema and burning after a second overnight application of a lotion containing 5% BPO on superficial acne lesions. (W. H. Eaglstein, Allergic Contact Dermatitis to Benzoyl Peroxide, Arch. Derm., 97, 527 (1968). In one report of allergic contact dermatitis to BPO resembling impetigo, the patient's dermatitis was exacerbated by the use of a 2.5% BP/1.2% clindamycin gel. (Kim et al, Allergic Contact Dermatitis to Benzoyl Peroxide Resembling Impetigo, Pediatric Dermatology, 32(4), e161-e162 (2015)).

Cases of patients being treated with BPO to treat skin conditions, resulting in diffuse facial erythema, swelling and aggravated papules, and an aggravation or complication of the rosacea conditions after such treatment, have been described. (Kim et al., Oral Azithromycin for Treatment of Intractable Rosacea, J. Korean. Med. Sci., 26, 694-96 (2011)). Minciullo et al. discusses a causation between angioedema in patients undergoing topical treatment of acne, and BPO-containing formulations used in such topical treatment. Minciullo et al., Allergic contact angioedema to benzoyl peroxide, J. Clin. Pharm. Therap., 31, 385-87 (2006)). Shwereb et al. describes an acute oedematous reaction to topical facial BPO application for acne. (Shwereb et al., Delayed Type Hypersensitivity to Benzoyl Peroxide, J. Drugs Dermatol., 3(2), 197-99 (2004)). Van Joost et al. describes the occurrence of purpuric contact dermatitis to BPO. (Van Joost et al., Purpuric contact dermatitis to benzoyl peroxide, J. Am. Acad. Dermatol., 22, 359-61 (1990)).

Such drawbacks associated with the treatment of rosacea involving the use of BPO result in exclusion of BPO from standard rosacea treatment methods. For example, “A Review of the Current Modalities for the Treatment of Papulopustular Rosacea” identifies metronidazole, ivermectin and azelaic acid as topical therapies that were proven effective for the treatment of rosacea. (McGregor et al., “A Review of the Current Modalities of the Treatment of Papulopustular Rosacea,” Dermatol. Clin. (2017)). While McGregor 2017 mentions alternate therapies, such as sodium sulfacetanide/sulfur cream, clindamycin, tretinoin, calcineurin inhibitors and oral tretinoin, that may have some effectiveness in the treatment of rosacea, notably, McGregor 2017 does not include, or even mention, BPO in the long list of possible treatment therapies described therein. The absence of BPO as a known treatment for rosacea is also evident in other studies. (Feaster et al., “Clinical effectiveness of novel rosacea therapies,” Current Op. Pharmacol., 46, 14-18 (2019); Del Rosso et al., “Update on the Management of Rosacea from the American Acne & Rosacea Society (AARS); J. Clinical & Aesthetic Dermat., 12 (6), 17-24 (2019)). The absence of BPO as a recognized first-line treatment for rosacea is especially evident in Del Rosso, which is a well-known and respected authority on the treatment of rosacea. The AARS review lists the Society's recommendation for rosacea treatment, including topical metronidazole, topical azelaic acid, oral tetracyclines, ivermectin, topical alpha agonists, and oral isotretinoin, as well as “alternative therapies,” such as sulfacetamide/sulfur, calcineurin inhibitors, retinoids, and permethrin. (See e.g., Table 1 of the AARS review.) BPO is not mentioned in the AARS review either as a leading, or even an alternative, therapeutic agent for the treatment of rosacea.

Rosacea is a chronic condition that seldom clears up without treatment, and its symptoms generally worsen with age. For example, as rosacea progresses, a subject's face can become more reddish, and blood vessels may become more visible. Onset of rosacea typically occurs between the ages of 30 and 50 years. (Huynh (2013) Am. Health Drug Benefits 6(6):348-354). Because rosacea symptoms can initially be mistaken for a blush or sunburn, treatment can be delayed, as patients may not be diagnosed until after progression of the disease and an increase in severity and/or duration of symptoms.

Compared to younger adult patients (e.g., patients aged 18-34), patents aged 35-64 years are more likely to have had increased cumulative exposure to one or more rosacea triggers that can increase the frequency and/or severity of rosacea symptoms. Rosacea triggers can include, for example, heat, cold, humidity, wind, sun exposure, alcohol, spicy foods, hot foods and drinks, exercise, stress, anxiety, certain skin products, certain medications (e.g., topical steroids, blood pressure medications, opiate painkillers, or combinations thereof), or combinations thereof, and the like.

Compared to younger adult patients (e.g., patients aged 18-34), patents aged 35-64 years are also more likely to have had increased exposure to intrinsic and extrinsic factors that can contribute to skin aging, which can exacerbate symptoms of rosacea. Intrinsic factors include atrophy of the dermis due to loss of collagen, degeneration in the elastic fiber network, and loss of hydration. Elastin and collagen, which help maintain structural integrity of blood vessel walls, decrease as people age. After around age 30, collagen content of skin decreases by about 1% per year. (Reilly et al. (2021) Plast. Aesthet. Res. 8(2)). The rate of elastin biosynthesis is relatively stable until around the third or fourth decade, after which it steeply declines. (Uitto (2008) J. Drugs Dermatol. 7(2): Supplement; Fazio et al. (1988) 58:270-277).

Extrinsic factors that can contribute to skin aging include UV exposure (photoaging), tobacco smoking, and alcohol. (Kohl et al. (2011) JEADV 25:873-884). UV exposure can change elastin metabolism, and result in accumulation of abnormal elastotic material. (Uitto (2008) J. Drugs Dermatol. 7(2): Supplement). Elastotic material can include elastin, fibrillin, glycosaminoglycans, particularly hyaluronic acid, and versican. (Kohl et al. (2011) JEADV 25:873-884). UV radiation can also generate reactive oxygen species (ROS), which can enhance inflammatory reactions and degenerate collagens and matrix in dermis. Matrix metalloproteinases (MMPs), which can digest dermal matrixes, such as collagens, fibronectin, and elastin, are inducible by UV irradiation and ROS stimulation. (Yamasaki et al. (2009) J. Dermatol. Sci. 55(2):77-81). Photodamage-induced collagen degeneration is associated with increased microvessel size and density. Decreased collagen is associated with symptoms of rosacea. In one study, patients with rosacea had significantly less collagen content than controls. Dermatology Times (2021) 42(4):26.

Considering the chronic nature of rosacea, there is a need for safe and effective treatments in patients who are more likely to exhibit rosacea symptoms with greater frequency and/or severity. For example, there is a need for safe and effective treatments in patients who are more likely to exhibit rosacea symptoms of greater frequency and/or severity due to effects skin aging (e.g., from intrinsic and/or extrinsic factors), greater exposure to rosacea triggers, a longer course of progression for the disease, or a combination thereof. In particular, there is a need for safe and effective treatments in patients who are aged 35-64. Compared to younger patients, patients who are aged 35-64 tend to have increased skin aging, a longer disease duration, and a greater cumulative exposure to rosacea triggers. For example, with aging in patients who are aged 35-64, the outer skin layer (epidermis) can thin, even though the number of cell layers can remain unchanged. A contributing factor to this can be loss of collagen. The number of pigment-containing cells (melanocytes) can decrease. Melanocytes can increase in size. With aging in patients who are aged 35-64, skin can be thinner, paler, clear, or any combination thereof, and the like. Changes in the connective tissue can reduce the skin's strength and elasticity. Elastosis, caused by changes in elastin production, can reduce skin's elastic quality. With aging in patients who are aged 35-64, blood vessels of the dermis can become more fragile, which can lead to bruising, bleeding under the skin (e.g., senile purpura). With aging in patients who are aged 35-64, the Sebaceous glands can produce less oil, which can result in dryness and/or itchiness. In patients who are aged 35-64, dry and/or sensitive skin can be more susceptible to adverse events from, and/or intolerance to, irritants, such as BPO. A non-limiting list of adverse events includes: irritation, dryness, scaling, itching purities, burning, stinging, combinations thereof and the like. Thus, treatment of rosacea patients aged 35-64 can be more challenging than treatment of younger rosacea patients.

Also, there is a need for early onset of action, and a prolonged use and/or treatment of the disease, its symptoms and associated conditions, in a safe and effective manner. Thus, there exists a need for compositions that show early onset of action, and improved efficacy in the treatment of rosacea, that impart greater tolerance to the active principles and that reduce, substantially minimize or do not have the side effects described in the prior art.

Advantages and features of the present disclosure, and methods for accomplishing the same will be more clearly understood from exemplary embodiments described below with reference to any accompanying drawings and figures. However, the present disclosure is not limited to the following exemplary embodiments and can be implemented in various different forms. The exemplary embodiments are provided only to provide sufficient disclosure of the present discoveries and to fully provide a person having ordinary skill in the art to which the present disclosure pertains within the technical field, and the present disclosure will be defined by any appended claims and combinations thereof.

As used herein, like reference numerals generally denote like elements throughout the present specification. Further, in the following description, a detailed explanation of well-known related technologies can be omitted to avoid unnecessarily obscuring the subject matter of the present disclosure.

As used herein, terms such as “including” and “having” are generally intended to allow other components to be included unless the terms are used in conjunction with the term “only.”

As used herein, the term “topical use” is meant to encompass the topical administration of an exemplary composition by formulating said composition in any way known in the art, or in formulations disclosed herein, which are compatible with the skin, mucous membranes and/or the integuments.

As used herein, the term “treating” or “treatment” includes curing a condition, treating a condition, preventing or substantially preventing a condition, treating symptoms of a condition, curing symptoms of a condition, ameliorating, reducing and/or minimizing symptoms of a condition, treating effects of a condition, ameliorating, reducing and/or minimizing effects of a condition, and preventing and/or substantially preventing results of a condition.

As used herein, the term “first-line therapy” or “first-line treatment” means a therapy or treatment for which its label does not include a requirement or recommendation that said therapy or treatment should be used only after other therapies or treatments were shown to be unsatisfactory or unsuccessful. It can also include a therapy and/or treatment wherein no other actives (beyond the main active) are administered to the individual subject in need. The term “first-line therapy” or “first-line treatment” can also include therapy or treatment in a subject who has not received prior therapy or treatment.

As used herein, the term “success rate” corresponds to a percentage of subjects achieving clear or almost clear skin on the investigator global assessment (IGA) scale after treatment with the pharmaceutical composition.

As used herein, the term “early onset” or “early onset of action” means achieving a desired result and/or effect at a point in time that is earlier or even much early than achieved using a vehicle or other, conventional treatment approach. For example, it can mean achieving a desired result and/or effect no later than about 8 weeks from initial treatment, preferably no later than about 4 weeks from initial treatment, and more preferably no later than about 2 weeks from initial treatment.

As used herein, the term “pharmaceutical composition” refers to a composition comprising one or more active ingredients with other components such as, for example, pharmaceutically acceptable ingredients and/or excipients. The purpose of a pharmaceutical composition is to facilitate administration of an active ingredient to a subject.

As used herein, the terms “pharmaceutically active agent” or “active agent” or “active pharmaceutical ingredient” are interchangeable and mean the ingredient is a pharmaceutical drug, which is biologically- and/or chemically-active and is regulatory-approved or approvable as such.

As used herein, the term “ingredient” refers to a pharmaceutically acceptable ingredient, which is included or is amenable to be included in The FDA's Inactive Ingredient (IIG) database. Inactive ingredients can sometimes exhibit some therapeutic effects, although they are not drugs.

As used herein, the term “adverse events values” refers to an average percentage of subjects that experience any adverse events associated with the treatment of rosacea with a composition described and/or claimed herein (usually on a surface of the skin of a subject treated with a composition described and/or claimed herein). A non-limiting list of such adverse events includes: irritation, dryness, scaling, itching purities, burning, stinging, combinations thereof and the like.

As used herein, the term “inflammatory lesion” refers to papules and pustules present on the skin of a patient, and does not include nodules and cysts.

As used herein, the term “papule” refers to a solid, elevated inflammatory lesion equal to or less than about 5 mm in diameter.

As used herein, the term “pustule” refers to an elevated inflammatory, pus-containing lesion equal to or less than about 5 mm in diameter.

As used herein, the term “nodule” and/or “cyst” refers to palpable solid inflammatory lesion, greater than about 5 mm in diameter. The nodule and/or cyst may have depth but does not necessarily include elevation.

Whenever a numerical range is indicated herewith, it is meant to include any cited numeral (fractional or integral) within the indicated range. The phrases “ranging/ranges between” a first indicated number and a second indicated number and “ranging/ranges from” a first indicated number “to” a second indicated number are used herein interchangeable and are meant to include the first and second indicated numbers and all fractional and integral numerals therebetween.

Reference to “at least about” a value, such as a percentage or a period of time, is understood to include “about” the value. For example, the phrases “at least about 2 weeks”, “at least about 4 weeks”, “at least about 8 weeks”, and “at least about 12 weeks” are understood to include “about 2 weeks”, “about 4 weeks”, “about 8 weeks” and “about 12 weeks”, respectively.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm.”

As used herein, numbers and/or numerical ranges preceded by the term “about” should not be considered to be limited to the recited range. Rather, numbers and/or numerical ranges preceded by the term “about” should be understood to include a range accepted by those skilled in the art for any given element in formations according to the subject invention.

As used herein, when a numerical value is preceded by the term “about,” the term “about” is intended to indicate +/−10%.

As used herein, the singular form “a,” “an” and “the” include plural references unless the context clearly dictates otherwise. For example, the term “a compound” or “at least one compound” can include a plurality of compounds, including combinations and/or mixtures thereof.

As used herein, the term “method” refers to manners, means, techniques and procedures for accomplishing a given task including, but not limited to, those manners, means, technical and procedures either known to, or readily developed from known manners, means, techniques and procedures by practitioners of the chemical, pharmacological, biological, biochemical and medical arts.

It is appreciated that certain features of the exemplary embodiments described herein, which are, for clarity, described in the context of separate embodiments, can also be provided in combination in a single embodiment. Conversely, various features of the exemplary embodiments, which are, for brevity, described in the context of a single embodiment, can also be provided separately or in any suitable sub-combination or as suitable in any other described embodiment. Certain features described in the context of various embodiments are not to be considered essential features of those embodiments, unless the embodiment is inoperative without those elements.

An exemplary embodiment of this application is a regimen for the therapeutic treatment of rosacea in a subject aged 35-64 years, the regimen comprising topically applying to the skin of a subject in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks (e.g., about 2 weeks), to achieve, in a group of such subjects, a success rate of at least about 12%, wherein the success rate is defined as the number of subjects achieving clear or almost clear skin on the investor global assessment (IGA) scale after treatment with the pharmaceutical composition. In other exemplary embodiments, the success rate after at least about 2 weeks (e.g., about 2 weeks) is about 12% to about 100%, about 12% to about 95%, about 12% to about 90%, about 12% to about 80%, about 12% to about 70%, about 12% to about 60%, about 12% to about 50%, about 12% to about 40%, or about 12% to about 30%.

In another exemplary embodiment, the success rate after at least about 2 weeks (e.g., about 2 weeks) is at least about 13%. In another exemplary embodiment, the success rate after at least about 2 weeks (e.g., about 2 weeks) is at least about 15%, at least about 20%, at least about 25%, or at least about 30%. In other exemplary embodiments, the success rate after at least about 2 weeks (e.g., about 2 weeks) is about 13% to about 100%, about 13% to about 95%, about 13% to about 90%, about 13% to about 80%, about 13% to about 70%, about 13% to about 60%, about 13% to about 50%, about 13% to about 40%, or about 13% to about 30%. In other exemplary embodiments, the success rate after at least about 2 weeks (e.g., about 2 weeks) is about 15% to about 100%, about 15% to about 95%, about 15% to about 90%, about 15% to about 80%, about 15% to about 70%, about 15% to about 60%, about 15% to about 50%, about 15% to about 40%, or about 15% to about 30%. In other exemplary embodiments, the success rate after at least about 2 weeks (e.g., about 2 weeks) is about 20% to about 100%, about 20% to about 95%, about 20% to about 90%, about 20% to about 80%, about 20% to about 70%, about 20% to about 60%, about 20% to about 50%, about 20% to about 40%, or about 20% to about 30%. In other exemplary embodiments, the success rate after at least about 2 weeks (e.g., about 2 weeks) is about 25% to about 100%, about 25% to about 95%, about 25% to about 90%, about 25% to about 80%, about 25% to about 70%, about 25% to about 60%, about 25% to about 50%, about 25% to about 40%, or about 25% to about 30%. In other exemplary embodiments, the success rate after at least about 2 weeks (e.g., about 2 weeks) is about 30% to about 100%, about 30% to about 95%, about 30% to about 90%, about 30% to about 80%, about 30% to about 70%, about 30% to about 60%, about 30% to about 50%, or about 30% to about 40%.

In another exemplary embodiment, the success rate of said regimen after treatment for at least about 2 weeks (e.g., about 2 weeks) of treatment is at least about 8% greater than a success rate achieved by a vehicle control (e.g., a vehicle cream). In another exemplary embodiment, the success rate after at least about 2 weeks (e.g., about 2 weeks) of treatment is at least about 10% greater than a success rate achieved by a vehicle control, at least about 15% greater than a success rate achieved by a vehicle control, at least about 20% greater than a success rate achieved by a vehicle control or at least about 25% greater than a success rate achieved by a vehicle control. In other exemplary embodiments, the success rate after at least about 2 weeks (e.g., about 2 weeks) of treatment is about 8% to about 70% greater than a success rate achieved by a vehicle control, about 8% to about 60% greater than a success rate achieved by a vehicle control, about 8% to about 50% greater than a success rate achieved by a vehicle control, about 8% to about 40% greater than a success rate achieved by a vehicle control, or about 8% to about 30% greater than a success rate achieved by a vehicle control. In other exemplary embodiments, the success rate after at least about 2 weeks (e.g., about 2 weeks) of treatment is about 10% to about 70% greater than a success rate achieved by a vehicle control, about 10% to about 60% greater than a success rate achieved by a vehicle control, about 10% to about 50% greater than a success rate achieved by a vehicle control, about 10% to about 40% greater than a success rate achieved by a vehicle control, or about 10% to about 30% greater than a success rate achieved by a vehicle control. In other exemplary embodiments, the success rate after at least about 2 weeks (e.g., about 2 weeks) of treatment is about 15% to about 70% greater than a success rate achieved by a vehicle control, about 15% to about 60% greater than a success rate achieved by a vehicle control, about 15% to about 50% greater than a success rate achieved by a vehicle control, about 15% to about 40% greater than a success rate achieved by a vehicle control, or about 15% to about 30% greater than a success rate achieved by a vehicle control. In other exemplary embodiments, the success rate after at least about 2 weeks (e.g., about 2 weeks) of treatment is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 2 weeks (e.g., about 2 weeks) of treatment is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control.

Another exemplary embodiment of this application is a regimen for the therapeutic treatment of rosacea in a subject aged 35-64 years, the regimen comprising topically applying to the skin of a subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks (e.g., about 4 weeks), to achieve, in a group of such subjects, a success rate of at least about 27%, wherein the success rate is defined as the number of subjects achieving clear or almost clear skin on the investor global assessment (IGA) scale after treatment with the pharmaceutical composition. In other exemplary embodiments, the success rate after at least about 4 weeks (e.g., about 4 weeks) of treatment is about 27% to about 100%, about 27% to about 95%, about 27% to about 90%, about 27% to about 80%, about 27% to about 70%, about 27% to about 60%, about 27% to about 50%, about 27% to about 40%, or about 27% to about 30%.

In another exemplary embodiment, the success rate after at least about 4 weeks (e.g., about 4 weeks) of treatment is at least about 30%. In another exemplary embodiment, the success rate after at least about 4 weeks (e.g., about 4 weeks) of treatment is at least about 35%, at least about 40%, at least about 45%, or at least about 50%. In other exemplary embodiments, the success rate after at least about 4 weeks (e.g., about 4 weeks) of treatment is about 30% to about 100%, about 30% to about 95%, about 30% to about 90%, about 30% to about 80%, about 30% to about 70%, about 30% to about 60%, about 30% to about 50%, or about 30% to about 40%. In other exemplary embodiments, the success rate after at least about 4 weeks (e.g., about 4 weeks) of treatment is about 35% to about 100%, about 35% to about 95%, about 35% to about 90%, about 35% to about 80%, about 35% to about 70%, about 35% to about 60%, about 35% to about 50%, or about 35% to about 40%. In other exemplary embodiments, the success rate after at least about 4 weeks (e.g., about 4 weeks) of treatment is about 40% to about 100%, about 40% to about 95%, about 40% to about 90%, about 40% to about 80%, about 40% to about 70%, about 40% to about 60%, or about 40% to about 50%. In other exemplary embodiments, the success rate after at least about 4 weeks (e.g., about 4 weeks) of treatment is about 45% to about 100%, about 45% to about 95%, about 45% to about 90%, about 45% to about 80%, about 45% to about 70%, about 45% to about 60%, or about 45% to about 50%. In other exemplary embodiments, the success rate after at least about 4 weeks (e.g., about 4 weeks) of treatment is about 50% to about 100%, about 50% to about 95%, about 50% to about 90%, about 50% to about 80%, about 50% to about 70%, or about 50% to about 60%.

In another exemplary embodiment, the success rate of said regimen after treatment for at least about 4 weeks (e.g., about 4 weeks) is at least about 17% greater than a success rate achieved by a vehicle control (e.g., a vehicle cream). In another exemplary embodiment, the success rate after at least about 4 weeks (e.g., about 4 weeks) of treatment is at least about 20% greater than a success rate achieved by a vehicle control, at least about 25% greater than a success rate achieved by a vehicle control, at least about 30% greater than a success rate achieved by a vehicle control or at least about 35% greater than a success rate achieved by a vehicle control. In other exemplary embodiments, the success rate after at least about 4 weeks (e.g., about 4 weeks) of treatment is about 17% to about 70% greater than a success rate achieved by a vehicle control, about 17% to about 60% greater than a success rate achieved by a vehicle control, about 17% to about 50% greater than a success rate achieved by a vehicle control, about 17% to about 40% greater than a success rate achieved by a vehicle control, or about 17% to about 30% greater than a success rate achieved by a vehicle control. In other exemplary embodiments, the success rate after at least about 4 weeks (e.g., about 4 weeks) of treatment is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 4 weeks (e.g., about 4 weeks) of treatment is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 4 weeks (e.g., about 4 weeks) of treatment is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 4 weeks (e.g., about 4 weeks) of treatment is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control.

Another exemplary embodiment of this application is a regimen for the therapeutic treatment of rosacea in a subject aged 35-64 years, the regimen comprising topically applying to the skin of the subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks (e.g., about 8 weeks), to achieve, in a group of such subjects, a success rate of at least about 38%, wherein the success rate is defined as the number of subjects achieving clear or almost clear skin on the investor global assessment (IGA) scale after treatment with the pharmaceutical composition. In other exemplary embodiments, the success rate after at least about 8 weeks (e.g., about 8 weeks) of treatment is about 38% to about 100%, about 38% to about 95%, about 38% to about 90%, about 38% to about 80%, about 38% to about 70%, about 38% to about 60%, about 38% to about 50%, or about 38% to about 40%.

In another exemplary embodiment, the success rate after at least about 8 weeks (e.g., about 8 weeks) of treatment is at least about 42%. In another exemplary embodiment, the success rate after at least about 8 weeks (e.g., about 8 weeks) of treatment is at least about 45%, at least about 50%, at least about 55%, or at least about 60%. In other exemplary embodiments, the success rate after at least about 8 weeks (e.g., about 8 weeks) of treatment is about 42% to about 100%, about 42% to about 95%, about 42% to about 90%, about 42% to about 80%, about 42% to about 70%, about 42% to about 60%, or about 42% to about 50%. In other exemplary embodiments, the success rate after at least about 8 weeks (e.g., about 8 weeks) of treatment is about 45% to about 100%, about 45% to about 95%, about 45% to about 90%, about 45% to about 80%, about 45% to about 70%, about 45% to about 60%, or about 45% to about 50%. In other exemplary embodiments, the success rate after at least about 8 weeks (e.g., about 8 weeks) of treatment is about 50% to about 100%, about 50% to about 95%, about 50% to about 90%, about 50% to about 80%, about 50% to about 70%, or about 50% to about 60%. In other exemplary embodiments, the success rate after at least about 8 weeks (e.g., about 8 weeks) of treatment is about 55% to about 100%, about 55% to about 95%, about 55% to about 90%, about 55% to about 80%, about 55% to about 70%, or about 55% to about 60%. In other exemplary embodiments, the success rate after at least about 8 weeks (e.g., about 8 weeks) of treatment is about 60% to about 100%, about 60% to about 95%, about 60% to about 90%, about 60% to about 80%, or about 60% to about 70%.

In another exemplary embodiment, the success rate of said regimen after at least about 8 weeks (e.g., about 8 weeks) of treatment is at least about 17% greater than a success rate achieved by the vehicle control (e.g., a vehicle cream). In another exemplary embodiment, the success rate after at least about 8 weeks (e.g., about 8 weeks) of treatment is at least about 20% greater than a success rate achieved by a vehicle control, at least about 25% greater than a success rate achieved by a vehicle control, at least about 30% greater than a success rate achieved by a vehicle control or at least about 35% greater than a success rate achieved by a vehicle control. In other exemplary embodiments, the success rate after at least about 8 weeks (e.g., about 8 weeks) of treatment is about 17% to about 70% greater than a success rate achieved by a vehicle control, about 17% to about 60% greater than a success rate achieved by a vehicle control, about 17% to about 50% greater than a success rate achieved by a vehicle control, about 17% to about 40% greater than a success rate achieved by a vehicle control, or about 17% to about 30% greater than a success rate achieved by a vehicle control. In other exemplary embodiments, the success rate after at least about 8 weeks (e.g., about 8 weeks) of treatment is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 8 weeks (e.g., about 8 weeks) of treatment is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 8 weeks (e.g., about 8 weeks) of treatment is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 8 weeks (e.g., about 8 weeks) of treatment is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control.

Another exemplary embodiment is a regimen for the therapeutic treatment of rosacea in a subject aged 35-64 years, the regimen comprising topically applying to the skin of the subject aged 35-64 years in need of said treatment a pharmaceutical composition, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks (e.g., about 12 weeks), to achieve, in a group of such subjects, a success rate of at least about 42%, wherein the success rate is defined as the number of subjects achieving clear or almost clear skin on the investor global assessment (IGA) scale after treatment with the pharmaceutical composition. In other exemplary embodiments, the success rate after at least about 12 weeks (e.g., about 12 weeks) of treatment is about 42% to about 100%, about 42% to about 95%, about 42% to about 90%, about 42% to about 80%, about 42% to about 70%, about 42% to about 60%, or about 42% to about 50%.

In another exemplary embodiment, the success rate after treatment for at least about 12 weeks (e.g., about 12 weeks) is at least about 47%. In another exemplary embodiment, the success rate after at least about 12 weeks (e.g., about 12 weeks) of treatment is at least about 50%, at least about 55%, at least about 60%, or at least about 65%. In other exemplary embodiments, the success rate after at least about 12 weeks (e.g., about 12 weeks) of treatment is about 47% to about 100%, about 47% to about 95%, about 47% to about 90%, about 47% to about 80%, about 47% to about 70%, about 47% to about 60%, or about 47% to about 50%. In other exemplary embodiments, the success rate after at least about 12 weeks (e.g., about 12 weeks) of treatment is about 50% to about 100%, about 50% to about 95%, about 50% to about 90%, about 50% to about 80%, about 50% to about 70%, or about 50% to about 60%. In other exemplary embodiments, the success rate after at least about 12 weeks (e.g., about 12 weeks) of treatment is about 55% to about 100%, about 55% to about 95%, about 55% to about 90%, about 55% to about 80%, about 55% to about 70%, or about 55% to about 60%. In other exemplary embodiments, the success rate after at least about 12 weeks (e.g., about 12 weeks) of treatment is about 60% to about 100%, about 60% to about 95%, about 60% to about 90%, about 60% to about 80%, or about 60% to about 70%. In other exemplary embodiments, the success rate after at least about 12 weeks (e.g., about 12 weeks) of treatment is about 65% to about 100%, about 65% to about 95%, about 65% to about 90%, about 65% to about 80%, or about 65% to about 70%.

In another exemplary embodiment, the success rate of said regimen after at least about 12 weeks (e.g., about 12 weeks) of treatment, is at least about 22% greater than the success rate achieved by the vehicle control (e.g., a vehicle cream). In another exemplary embodiment, the success rate after at least about 12 weeks (e.g., about 12 weeks) of treatment is at least about 25% greater than a success rate achieved by a vehicle control, at least about 30% greater than a success rate achieved by a vehicle control, at least about 35% greater than a success rate achieved by a vehicle control or at least about 40% greater than a success rate achieved by a vehicle control. In other exemplary embodiments, the success rate after at least about 12 weeks (e.g., about 12 weeks) of treatment is about 22% to about 70% greater than a success rate achieved by a vehicle control, about 22% to about 60% greater than a success rate achieved by a vehicle control, about 22% to about 50% greater than a success rate achieved by a vehicle control, about 22% to about 40% greater than a success rate achieved by a vehicle control, or about 22% to about 30% greater than a success rate achieved by a vehicle control. In other exemplary embodiments, the success rate after at least about 12 weeks (e.g., about 12 weeks) of treatment is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 12 weeks (e.g., about 12 weeks) of treatment is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 12 weeks (e.g., about 12 weeks) of treatment is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control. In other exemplary embodiments, the success rate after at least about 12 weeks (e.g., about 12 weeks) of treatment is about 40% to about 70% greater than a success rate achieved by a vehicle control, about 40% to about 60% greater than a success rate achieved by a vehicle control, or about 40% to about 50% greater than a success rate achieved by a vehicle control.

In another exemplary embodiment the success rate of said regimen, for a subject aged 35-64 years and after treatment for at least about 2 weeks (e.g., about 2 weeks), at least about 4 weeks (e.g., about 4 weeks), at least about 8 weeks (e.g., about 8 weeks), or at least about 12 weeks (e.g., about 12 weeks) with the pharmaceutical composition, is at least about 7% greater than a success rate achieved after treatment of a subject aged 18-35 years for at least about 2 weeks (e.g., about 2 weeks), at least about 4 weeks (e.g., about 4 weeks), at least about 8 weeks (e.g., about 8 weeks), or at least about 12 weeks (e.g., about 12 weeks), respectively, with the pharmaceutical composition. In another exemplary embodiment, the success rate of said regimen, for a subject aged 35-64 years, after treatment for at least about 2 weeks (e.g., about 2 weeks), at least about 4 weeks (e.g., about 4 weeks), at least about 8 weeks (e.g., about 8 weeks), or at least about 12 weeks (e.g., about 12 weeks) with the pharmaceutical composition, is at least about 8% greater, at least about 10% greater, at least about 12% greater, at least about 15% greater, about 7% to about 60% greater, about 8% to about 60% greater, about 10% to about 60% greater, about 12% to about 60% greater, about 15% to about 60% greater, about 7% to about 50% greater, about 8% to about 50% greater, about 10% to about 50% greater, about 12% to about 50% greater, about 15% to about 50% greater, about 7% to about 40% greater, about 8% to about 40% greater, about 10% to about 40% greater, about 12% to about 40% greater, about 15% to about 40% greater, about 7% to about 30% greater, about 8% to about 30% greater, about 10% to about 30% greater, about 12% to about 30% greater, or about 15% to about 30% greater, than a success rate achieved after treatment of a subject aged 18-35 years for at least about 2 weeks, at least about 4 weeks, at least about 8 weeks, or at least about 12 weeks, respectively, with the pharmaceutical composition.

In other exemplary embodiments, the success rate of said regimen, for subjects aged 35-64 years and after treatment for at least about 2 weeks, at least about 4 weeks, at least about 8 weeks, or at least about 12 weeks with the pharmaceutical composition, is at least about 3% greater, at least about 4% greater, at least about 5% greater, at least about 6% greater, about 3% to about 60% greater, about 3% to about 50% greater, about 3% to about 40% greater, about 3% to about 30% greater, about 4% to about 60% greater, about 4% to about 50% greater, about 4% to about 40% greater, about 4% to about 30% greater, about 5% to about 60% greater, about 5% to about 50% greater, about 5% to about 40% greater, about 5% to about 30% greater, about 6% to about 60% greater, about 6% to about 50% greater, about 6% to about 40% greater, or about 6% to about 30% greater, than a success rate achieved after treatment of subjects aged 18-35 years for at least about 2 weeks, at least about 4 weeks, at least about 8 weeks, or at least about 12 weeks, respectively, with the pharmaceutical composition.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks (e.g., about 2 weeks), to achieve, in a group of such subjects, a mean percentage decrease, from baseline, in the number of inflammatory lesions of at least about 38%. In other exemplary embodiments, the mean percentage decrease in the number of inflammatory lesions after at least about 2 weeks (e.g., about 2 weeks) is about 38% to about 100%, about 38% to about 95%, about 38% to about 90%, about 38% to about 80%, about 38% to about 70%, about 38% to about 60%, about 38% to about 50%, or about 38% to about 40%.

In another exemplary embodiment, the mean percentage decrease in the number of inflammatory lesions after at least about 2 weeks (e.g., about 2 weeks) is at least about 42%. In another exemplary embodiment, the mean percentage decrease in the number of inflammatory lesions after at least about 2 weeks (e.g., about 2 weeks) is at least about 45%, at least about 50%, at least about 55%, or at least about 60%. In other exemplary embodiments, the mean percentage decrease in the number of inflammatory lesions after at least about 2 weeks (e.g., about 2 weeks) is about 42% to about 100%, about 42% to about 95%, about 42% to about 90%, about 42% to about 80%, about 42% to about 70%, about 42% to about 60%, or about 42% to about 50%. In other exemplary embodiments, the mean percentage decrease in the number of inflammatory lesions after at least about 2 weeks (e.g., about 2 weeks) is about 45% to about 100%, about 45% to about 95%, about 45% to about 90%, about 45% to about 80%, about 45% to about 70%, about 45% to about 60%, or about 45% to about 50%. In other exemplary embodiments, the mean percentage decrease in the number of inflammatory lesions after at least about 2 weeks (e.g., about 2 weeks) is about 50% to about 100%, about 50% to about 95%, about 50% to about 90%, about 50% to about 80%, about 50% to about 70%, or about 50% to about 60%. In other exemplary embodiments, the mean percentage decrease in the number of inflammatory lesions after at least about 2 weeks (e.g., about 2 weeks) is about 55% to about 100%, about 55% to about 95%, about 55% to about 90%, about 55% to about 80%, about 55% to about 70%, or about 55% to about 60%. In other exemplary embodiments, the mean percentage decrease in the number of inflammatory lesions after at least about 2 weeks (e.g., about 2 weeks) is about 60% to about 100%, about 60% to about 95%, about 60% to about 90%, about 60% to about 80%, or about 60% to about 70%.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks (e.g., about 4 weeks), to achieve, in a group of such subjects, a mean percentage decrease, from baseline, in the number of inflammatory lesions of at least about 52%. In other exemplary embodiments, the mean percentage decrease in the number of inflammatory lesions after at least about 4 weeks (e.g., about 4 weeks) is about 52% to about 100%, about 52% to about 95%, about 52% to about 90%, about 52% to about 80%, about 52% to about 70%, or about 52% to about 60%.

In another exemplary embodiment, the mean percentage decrease in the number of inflammatory lesions after at least about 4 weeks (e.g., about 4 weeks) is at least about 58%. In another exemplary embodiment, the mean percentage decrease in the number of inflammatory lesions after at least about 4 weeks (e.g., about 4 weeks) is at least about 60%, at least about 65%, at least about 70%, or at least about 75%. In other exemplary embodiments, the mean percentage decrease in the number of inflammatory lesions after at least about 4 weeks (e.g., about 4 weeks) is about 58% to about 100%, about 58% to about 95%, about 58% to about 90%, about 58% to about 80%, about 58% to about 70%, or about 58% to about 60%. In other exemplary embodiments, the mean percentage decrease in the number of inflammatory lesions after at least about 4 weeks (e.g., about 4 weeks) is about 60% to about 100%, about 60% to about 95%, about 60% to about 90%, about 60% to about 80%, or about 60% to about 70%. In other exemplary embodiments, the mean percentage decrease in the number of inflammatory lesions after at least about 4 weeks (e.g., about 4 weeks) is about 65% to about 100%, about 65% to about 95%, about 65% to about 90%, about 65% to about 80%, or about 65% to about 70%. In other exemplary embodiments, the mean percentage decrease in the number of inflammatory lesions after at least about 4 weeks (e.g., about 4 weeks) is about 70% to about 100%, about 70% to about 95%, about 70% to about 90%, or about 70% to about 80%. In other exemplary embodiments, the mean percentage decrease in the number of inflammatory lesions after at least about 4 weeks (e.g., about 4 weeks) is about 75% to about 100%, about 75% to about 95%, about 75% to about 90%, or about 75% to about 80%.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks (e.g., about 8 weeks) to achieve, in a group of such subjects, a mean percentage decrease, from baseline, in the number of inflammatory lesions of at least about 61%. In other exemplary embodiments, the mean percentage decrease in the number of inflammatory lesions after at least about 8 weeks (e.g., about 8 weeks) is about 61% to about 100%, about 61% to about 95%, about 61% to about 90%, about 61% to about 80%, or about 61% to about 70%.

In another exemplary embodiment, the mean percentage decrease in the number of inflammatory lesions after at least about 8 weeks (e.g., about 8 weeks) is at least about 68%. In another exemplary embodiment, the mean percentage decrease in the number of inflammatory lesions after at least about 8 weeks (e.g., about 8 weeks) is at least about 70%, at least about 75%, at least about 80%, or at least about 85%. In other exemplary embodiments, the mean percentage decrease in the number of inflammatory lesions after at least about 8 weeks (e.g., about 8 weeks) is about 68% to about 100%, about 68% to about 95%, about 68% to about 90%, about 68% to about 80%, or about 68% to about 70%. In other exemplary embodiments, the mean percentage decrease in the number of inflammatory lesions after at least about 8 weeks (e.g., about 8 weeks) is about 70% to about 100%, about 70% to about 95%, about 70% to about 90%, or about 70% to about 80%. In other exemplary embodiments, the mean percentage decrease in the number of inflammatory lesions after at least about 8 weeks (e.g., about 8 weeks) is about 75% to about 100%, about 75% to about 95%, about 75% to about 90%, or about 75% to about 80%. In other exemplary embodiments, the mean percentage decrease in the number of inflammatory lesions after at least about 8 weeks (e.g., about 8 weeks) is about 80% to about 100%, about 80% to about 95%, or about 80% to about 90%. In other exemplary embodiments, the mean percentage decrease in the number of inflammatory lesions after at least about 8 weeks (e.g., about 8 weeks) is about 85% to about 100%, about 85% to about 95%, or about 85% to about 90%.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks (e.g., about 12 weeks), to achieve, in a group of such subjects, a mean percentage decrease, from baseline, in the number of inflammatory lesions of at least about 62%. In other exemplary embodiments, the mean percentage decrease in the number of inflammatory lesions after at least about 12 weeks (e.g., about 12 weeks) is about 62% to about 100%, about 62% to about 95%, about 62% to about 90%, about 62% to about 80%, or about 62% to about 70%.

In another exemplary embodiment, the mean percentage decrease in the number of inflammatory lesions after at least about 12 weeks (e.g., about 12 weeks) is at least about 69%. In another exemplary embodiment, the mean percentage decrease in the number of inflammatory lesions after at least about 12 weeks (e.g., about 12 weeks) is at least about 70%, at least about 75%, at least about 80%, or at least about 85%. In other exemplary embodiments, the mean percentage decrease in the number of inflammatory lesions after at least about 12 weeks (e.g., about 12 weeks) is about 69% to about 100%, about 69% to about 95%, about 69% to about 90%, about 69% to about 80%, or about 69% to about 70%. In other exemplary embodiments, the mean percentage decrease in the number of inflammatory lesions after at least about 12 weeks (e.g., about 12 weeks) is about 70% to about 100%, about 70% to about 95%, about 70% to about 90%, or about 70% to about 80%. In other exemplary embodiments, the mean percentage decrease in the number of inflammatory lesions after at least about 12 weeks (e.g., about 12 weeks) is about 75% to about 100%, about 75% to about 95%, about 75% to about 90%, or about 75% to about 80%. In other exemplary embodiments, the mean percentage decrease in the number of inflammatory lesions after at least about 12 weeks (e.g., about 12 weeks) is about 80% to about 100%, about 80% to about 95%, or about 80% to about 90%. In other exemplary embodiments, the mean percentage decrease in the number of inflammatory lesions after at least about 12 weeks (e.g., about 12 weeks) is about 85% to about 100%, about 85% to about 95%, or about 85% to about 90%.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks (e.g., about 2 weeks), to achieve, in a group of such subjects, a mean percentage decrease, from baseline, in the number of inflammatory lesions of at least about 42% compared to a mean percentage decrease, from baseline, in the inflammatory lesions of at least about 23% after treatment with vehicle control.

In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 23% after treatment with vehicle control.

In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 23% after treatment with vehicle control. In other exemplary embodiments, the mean percentage decrease in the number of inflammatory lesions after at least about 2 weeks (e.g., about 2 weeks) is about 40% to about 70% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 23% to about 25% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 4 weeks (e.g., about 4 weeks), to achieve, in a group of such subjects, a mean percentage decrease, from baseline, in the number of inflammatory lesions of at least about 58% compared to a mean percentage decrease, from baseline, in the inflammatory lesions of at least about 36% after treatment with vehicle control.

In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 36% after treatment with vehicle control.

In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 36% after treatment with vehicle control. In other exemplary embodiments, the mean percentage decrease in the number of inflammatory lesions after at least about 4 weeks (e.g., about 4 weeks) is about 55% to about 75% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 36% to about 38% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 8 weeks (e.g., about 8 weeks), to achieve, in a group of such subjects, a mean percentage decrease, from baseline, in the number of inflammatory lesions of at least about 68% compared to a mean percentage decrease, from baseline, in the inflammatory lesions of at least about 42% after treatment with vehicle control.

In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 42% after treatment with vehicle control.

In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 42% after treatment with vehicle control. In other exemplary embodiments, the mean percentage decrease in the number of inflammatory lesions after at least about 8 weeks (e.g., about 8 weeks) is about 65% to about 85% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 42% to about 44% after treatment with vehicle control.

Another exemplary embodiment is a regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 12 weeks (e.g., about 12 weeks), to achieve, in a group of such subjects, a mean percentage decrease, from baseline, in the number of inflammatory lesions of at least about 69% compared to a mean percentage decrease, from baseline, in the inflammatory lesions of at least about 41% after treatment with vehicle control.

In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 41% after treatment with vehicle control.

In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 41% after treatment with vehicle control. In other exemplary embodiments, the mean percentage decrease in the number of inflammatory lesions after at least about 12 weeks (e.g., about 12 weeks) is about 65% to about 85% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 41% to about 43% after treatment with vehicle control.

In other exemplary embodiments, the benzoyl peroxide is the sole active ingredient administered to the subject during the duration of the regimen.

In other exemplary embodiments, the pharmaceutical composition comprises about 2.5% w/w to about 10% w/w of benzoyl peroxide, preferably about 3% to about 9%, about 4% to about 8%, and more preferably about 5% w/w of benzoyl peroxide.

In other exemplary embodiments, the benzoyl peroxide is in a form selected from the group consisting of solid, solution, or suspension.

In other exemplary embodiments, the regimen is a first line therapy for the treatment of rosacea. In some exemplary embodiments, the regimen is a first line therapy for the treatment of rosacea in a subject aged 35-64 years.

In other exemplary embodiments, the rosacea is any of erythematotelangiectatic rosacea, papulopustular rosacea, phymatous rosacea, ocular rosacea, and combinations thereof.

In other exemplary embodiments, the rosacea is moderate to severe rosacea, preferably severe rosacea.

In other exemplary embodiments, the pharmaceutical composition is a cream or an emulsion.

In other exemplary embodiments, the pharmaceutical composition is an extended release formulation.

In other exemplary embodiments, the benzoyl peroxide is present in an inert system that provides a barrier between the benzoyl peroxide and its surrounding environment.

In other exemplary embodiments, the benzoyl peroxide is released in predetermined amounts over a specified period of time

In other exemplary embodiments, the extended-release effect is obtained by encapsulation, microencapsulation, microspheres, a coating, or a combination thereof, and the like.

In other exemplary embodiments, the benzoyl peroxide is encapsulated or micro encapsulated.

In other exemplary embodiments, the benzoyl peroxide is included in a microsphere or a coating.

In other exemplary embodiments, the regimen comprises topically applying the pharmaceutical composition to the skin of a subject aged 40-64 years, 45-64 years, 50-64 years, 55-64 years, or 60-64 years in need of said treatment. In other exemplary embodiments, the regimen comprises topically applying the pharmaceutical composition to the skin of a subject aged 35-60 years, 35-55 years, 35-50 years, 35-45 years, or 35-40 years in need of said treatment.

Another exemplary embodiment is a pharmaceutical composition for use in the treatment of rosacea (e.g., severe rosacea) in a subject aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, and said pharmaceutical composition is applied (or capable of being applied) once daily for a period of at least about 2 weeks (e.g., about 2 weeks), at least about 4 weeks (e.g., about 4 weeks), at least about 8 weeks (e.g., about 8 weeks) or at least about 12 weeks (e.g., about 12 weeks), to achieve, in a group of such subjects, a success rate of at least about 12%, at least about 27%, at least about 38%, or at least about 42%, respectively, wherein the success rate is defined as the number of subjects achieving clear or almost clear skin on the investor global assessment (IGA) scale after treatment with the pharmaceutical composition. In other exemplary embodiments, the success rate after at least about 2 weeks (e.g., about 2 weeks) is about 12% to about 100%, about 12% to about 95%, about 12% to about 90%, about 12% to about 80%, about 12% to about 70%, about 12% to about 60%, about 12% to about 50%, about 12% to about 40%, or about 12% to about 30%. In other exemplary embodiments, the success rate after at least about 4 weeks (e.g., about 4 weeks) is about 27% to about 100%, about 27% to about 95%, about 27% to about 90%, about 27% to about 80%, about 27% to about 70%, about 27% to about 60%, about 27% to about 50%, about 27% to about 40%, or about 27% to about 30%. In other exemplary embodiments, the success rate after at least about 8 weeks (e.g., about 8 weeks) is about 38% to about 100%, about 38% to about 95%, about 38% to about 90%, about 38% to about 80%, about 38% to about 70%, about 38% to about 60%, about 38% to about 50%, or about 38% to about 40%. In other exemplary embodiments, the success rate after at least about 12 weeks (e.g., about 12 weeks) is about 42% to about 100%, about 42% to about 95%, about 42% to about 90%, about 42% to about 80%, about 42% to about 70%, about 42% to about 60%, or about 42% to about 50%.

In other exemplary embodiments, the pharmaceutical composition is applied (or capable of being applied) once daily for a period of at least about 2 weeks (e.g., about 2 weeks), at least about 4 weeks (e.g., about 4 weeks), at least about 8 weeks (e.g., about 8 weeks) or at least about 12 weeks (e.g., about 12 weeks), to achieve, in a group of such subjects, a success rate of at least about 13%, at least about 30%, at least about 42%, or at least about 47%, respectively. In another exemplary embodiment, the success rate after at least about 2 weeks (e.g., about 2 weeks) is at least about 15%, at least about 20%, at least about 25%, or at least about 30%. In other exemplary embodiments, the success rate after at least about 2 weeks (e.g., about 2 weeks) is about 13% to about 100%, about 13% to about 95%, about 13% to about 90%, about 13% to about 80%, about 13% to about 70%, about 13% to about 60%, about 13% to about 50%, about 13% to about 40%, or about 13% to about 30%. In other exemplary embodiments, the success rate after at least about 2 weeks (e.g., about 2 weeks) is about 15% to about 100%, about 15% to about 95%, about 15% to about 90%, about 15% to about 80%, about 15% to about 70%, about 15% to about 60%, about 15% to about 50%, about 15% to about 40%, or about 15% to about 30%. In other exemplary embodiments, the success rate after at least about 2 weeks (e.g., about 2 weeks) is about 20% to about 100%, about 20% to about 95%, about 20% to about 90%, about 20% to about 80%, about 20% to about 70%, about 20% to about 60%, about 20% to about 50%, about 20% to about 40%, or about 20% to about 30%. In other exemplary embodiments, the success rate after at least about 2 weeks (e.g., about 2 weeks) is about 25% to about 100%, about 25% to about 95%, about 25% to about 90%, about 25% to about 80%, about 25% to about 70%, about 25% to about 60%, about 25% to about 50%, about 25% to about 40%, or about 25% to about 30%. In other exemplary embodiments, the success rate after at least about 2 weeks (e.g., about 2 weeks) is about 30% to about 100%, about 30% to about 95%, about 30% to about 90%, about 30% to about 80%, about 30% to about 70%, about 30% to about 60%, about 30% to about 50%, or about 30% to about 40%.

In another exemplary embodiment, the success rate after at least about 4 weeks (e.g., about 4 weeks) is at least about 35%, at least about 40%, at least about 45%, or at least about 50%. In other exemplary embodiments, the success rate after at least about 4 weeks (e.g., about 4 weeks) is about 30% to about 100%, about 30% to about 95%, about 30% to about 90%, about 30% to about 80%, about 30% to about 70%, about 30% to about 60%, about 30% to about 50%, or about 30% to about 40%. In other exemplary embodiments, the success rate after at least about 4 weeks (e.g., about 4 weeks) is about 35% to about 100%, about 35% to about 95%, about 35% to about 90%, about 35% to about 80%, about 35% to about 70%, about 35% to about 60%, about 35% to about 50%, or about 35% to about 40%. In other exemplary embodiments, the success rate after at least about 4 weeks (e.g., about 4 weeks) is about 40% to about 100%, about 40% to about 95%, about 40% to about 90%, about 40% to about 80%, about 40% to about 70%, about 40% to about 60%, or about 40% to about 50%. In other exemplary embodiments, the success rate after at least about 4 weeks (e.g., about 4 weeks) is about 45% to about 100%, about 45% to about 95%, about 45% to about 90%, about 45% to about 80%, about 45% to about 70%, about 45% to about 60%, or about 45% to about 50%. In other exemplary embodiments, the success rate after at least about 4 weeks (e.g., about 4 weeks) is about 50% to about 100%, about 50% to about 95%, about 50% to about 90%, about 50% to about 80%, about 50% to about 70%, or about 50% to about 60%.

In another exemplary embodiment, the success rate after at least about 8 weeks (e.g., about 8 weeks) is at least about 45%, at least about 50%, at least about 55%, or at least about 60%. In other exemplary embodiments, the success rate after at least about 8 weeks (e.g., about 8 weeks) is about 42% to about 100%, about 42% to about 95%, about 42% to about 90%, about 42% to about 80%, about 42% to about 70%, about 42% to about 60%, or about 42% to about 50%. In other exemplary embodiments, the success rate after at least about 8 weeks (e.g., about 8 weeks) is about 45% to about 100%, about 45% to about 95%, about 45% to about 90%, about 45% to about 80%, about 45% to about 70%, about 45% to about 60%, or about 45% to about 50%. In other exemplary embodiments, the success rate after at least about 8 weeks (e.g., about 8 weeks) is about 50% to about 100%, about 50% to about 95%, about 50% to about 90%, about 50% to about 80%, about 50% to about 70%, or about 50% to about 60%. In other exemplary embodiments, the success rate after at least about 8 weeks (e.g., about 8 weeks) is about 55% to about 100%, about 55% to about 95%, about 55% to about 90%, about 55% to about 80%, about 55% to about 70%, or about 55% to about 60%. In other exemplary embodiments, the success rate after at least about 8 weeks (e.g., about 8 weeks) is about 60% to about 100%, about 60% to about 95%, about 60% to about 90%, about 60% to about 80%, or about 60% to about 70%.

In another exemplary embodiment, the success rate after at least about 12 weeks (e.g., about 12 weeks) is at least about 47%. In another exemplary embodiment, the success rate after at least about 12 weeks (e.g., about 12 weeks) is at least about 50%, at least about 55%, at least about 60%, or at least about 65%. In other exemplary embodiments, the success rate after at least about 12 weeks (e.g., about 12 weeks) is about 47% to about 100%, about 47% to about 95%, about 47% to about 90%, about 47% to about 80%, about 47% to about 70%, about 47% to about 60%, or about 47% to about 50%. In other exemplary embodiments, the success rate after at least about 12 weeks (e.g., about 12 weeks) is about 50% to about 100%, about 50% to about 95%, about 50% to about 90%, about 50% to about 80%, about 50% to about 70%, or about 50% to about 60%. In other exemplary embodiments, the success rate after at least about 12 weeks (e.g., about 12 weeks) is about 55% to about 100%, about 55% to about 95%, about 55% to about 90%, about 55% to about 80%, about 55% to about 70%, or about 55% to about 60%. In other exemplary embodiments, the success rate after at least about 12 weeks (e.g., about 12 weeks) is about 60% to about 100%, about 60% to about 95%, about 60% to about 90%, about 60% to about 80%, or about 60% to about 70%. In other exemplary embodiments, the success rate after at least about 12 weeks (e.g., about 12 weeks) is about 65% to about 100%, about 65% to about 95%, about 65% to about 90%, about 65% to about 80%, or about 65% to about 70%.

In other exemplary embodiments, the success rate after at least about 2 weeks (e.g., about 2 weeks), at least about 4 weeks (e.g., about 4 weeks), at least about 8 weeks (e.g., about 8 weeks) or at least about 12 weeks (e.g., about 12 weeks), is at least about 8% greater, at least about 17% greater, at least about 17% greater, or at least about 22% greater, respectively, than a success rate achieved after treatment for at least about 2 weeks (e.g., about 2 weeks), at least about 4 weeks (e.g., about 4 weeks), at least about 8 weeks (e.g., about 8 weeks) or at least about 12 weeks (e.g., about 12 weeks), respectively, with a vehicle control.

In another exemplary embodiment, the success rate after at least about 2 weeks (e.g., about 2 weeks) is at least about 10% greater than a success rate achieved by a vehicle control, at least about 15% greater than a success rate achieved by a vehicle control, at least about 20% greater than a success rate achieved by a vehicle control or at least about 25% greater than a success rate achieved by a vehicle control. In other exemplary embodiments, the success rate after at least about 2 weeks (e.g., about 2 weeks) is about 8% to about 70% greater than a success rate achieved by a vehicle control, about 8% to about 60% greater than a success rate achieved by a vehicle control, about 8% to about 50% greater than a success rate achieved by a vehicle control, about 8% to about 40% greater than a success rate achieved by a vehicle control, or about 8% to about 30% greater than a success rate achieved by a vehicle control. In other exemplary embodiments, the success rate after at least about 2 weeks (e.g., about 2 weeks) is about 10% to about 70% greater than a success rate achieved by a vehicle control, about 10% to about 60% greater than a success rate achieved by a vehicle control, about 10% to about 50% greater than a success rate achieved by a vehicle control, about 10% to about 40% greater than a success rate achieved by a vehicle control, or about 10% to about 30% greater than a success rate achieved by a vehicle control. In other exemplary embodiments, the success rate after at least about 2 weeks (e.g., about 2 weeks) is about 15% to about 70% greater than a success rate achieved by a vehicle control, about 15% to about 60% greater than a success rate achieved by a vehicle control, about 15% to about 50% greater than a success rate achieved by a vehicle control, about 15% to about 40% greater than a success rate achieved by a vehicle control, or about 15% to about 30% greater than a success rate achieved by a vehicle control. In other exemplary embodiments, the success rate after at least about 2 weeks (e.g., about 2 weeks) is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 2 weeks (e.g., about 2 weeks) is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control.

In another exemplary embodiment, the success rate after at least about 4 weeks (e.g., about 4 weeks) is at least about 20% greater than a success rate achieved by a vehicle control, at least about 25% greater than a success rate achieved by a vehicle control, at least about 30% greater than a success rate achieved by a vehicle control or at least about 35% greater than a success rate achieved by a vehicle control. In other exemplary embodiments, the success rate after at least about 4 weeks (e.g., about 4 weeks) is about 17% to about 70% greater than a success rate achieved by a vehicle control, about 17% to about 60% greater than a success rate achieved by a vehicle control, about 17% to about 50% greater than a success rate achieved by a vehicle control, about 17% to about 40% greater than a success rate achieved by a vehicle control, or about 17% to about 30% greater than a success rate achieved by a vehicle control. In other exemplary embodiments, the success rate after at least about 4 weeks (e.g., about 4 weeks) is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 4 weeks (e.g., about 4 weeks) is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 4 weeks (e.g., about 4 weeks) is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 4 weeks (e.g., about 4 weeks) is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control.

In another exemplary embodiment, the success rate after at least about 8 weeks (e.g., about 8 weeks) is at least about 20% greater than a success rate achieved by a vehicle control, at least about 25% greater than a success rate achieved by a vehicle control, at least about 30% greater than a success rate achieved by a vehicle control or at least about 35% greater than a success rate achieved by a vehicle control. In other exemplary embodiments, the success rate after at least about 8 weeks (e.g., about 8 weeks) is about 17% to about 70% greater than a success rate achieved by a vehicle control, about 17% to about 60% greater than a success rate achieved by a vehicle control, about 17% to about 50% greater than a success rate achieved by a vehicle control, about 17% to about 40% greater than a success rate achieved by a vehicle control, or about 17% to about 30% greater than a success rate achieved by a vehicle control. In other exemplary embodiments, the success rate after at least about 8 weeks (e.g., about 8 weeks) is about 20% to about 70% greater than a success rate achieved by a vehicle control, about 20% to about 60% greater than a success rate achieved by a vehicle control, about 20% to about 50% greater than a success rate achieved by a vehicle control, about 20% to about 40% greater than a success rate achieved by a vehicle control, or about 20% to about 30% greater than a success rate achieved by a vehicle control. In other exemplary embodiments, the success rate after at least about 8 weeks (e.g., about 8 weeks) is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 8 weeks (e.g., about 8 weeks) is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 8 weeks (e.g., about 8 weeks) is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control.

In another exemplary embodiment, the success rate after at least about 12 weeks (e.g., about 12 weeks) is at least about 25% greater than a success rate achieved by a vehicle control, at least about 30% greater than a success rate achieved by a vehicle control, at least about 35% greater than a success rate achieved by a vehicle control or at least about 40% greater than a success rate achieved by a vehicle control. In other exemplary embodiments, the success rate after at least about 12 weeks (e.g., about 12 weeks) is about 22% to about 70% greater than a success rate achieved by a vehicle control, about 22% to about 60% greater than a success rate achieved by a vehicle control, about 22% to about 50% greater than a success rate achieved by a vehicle control, about 22% to about 40% greater than a success rate achieved by a vehicle control, or about 22% to about 30% greater than a success rate achieved by a vehicle control. In other exemplary embodiments, the success rate after at least about 12 weeks (e.g., about 12 weeks) is about 25% to about 70% greater than a success rate achieved by a vehicle control, about 25% to about 60% greater than a success rate achieved by a vehicle control, about 25% to about 50% greater than a success rate achieved by a vehicle control, about 25% to about 40% greater than a success rate achieved by a vehicle control, or about 25% to about 30% greater than a success rate achieved by a vehicle control. In other exemplary embodiments, the success rate after at least about 12 weeks (e.g., about 12 weeks) is about 30% to about 70% greater than a success rate achieved by a vehicle control, about 30% to about 60% greater than a success rate achieved by a vehicle control, about 30% to about 50% greater than a success rate achieved by a vehicle control, or about 30% to about 40% greater than a success rate achieved by a vehicle control. In other exemplary embodiments, the success rate after at least about 12 weeks (e.g., about 12 weeks) is about 35% to about 70% greater than a success rate achieved by a vehicle control, about 35% to about 60% greater than a success rate achieved by a vehicle control, about 35% to about 50% greater than a success rate achieved by a vehicle control, or about 35% to about 40% greater than a success rate achieved by a vehicle control. In other exemplary embodiments, the success rate after at least about 12 weeks (e.g., about 12 weeks) is about 40% to about 70% greater than a success rate achieved by a vehicle control, about 40% to about 60% greater than a success rate achieved by a vehicle control, or about 40% to about 50% greater than a success rate achieved by a vehicle control.

In other exemplary embodiments, the success rate, for subjects aged 35-64 years and after treatment for at least about 2 weeks (e.g., about 2 weeks), at least about 4 weeks (e.g., about 4 weeks), at least about 8 weeks (e.g., about 8 weeks), or at least about 12 weeks (e.g., about 12 weeks) with the pharmaceutical composition, is at least about 7% greater than a success rate achieved after treatment of subjects aged 18-35 years for at least about 2 weeks (e.g., about 2 weeks), at least about 4 weeks (e.g., about 4 weeks), at least about 8 weeks (e.g., about 8 weeks), or at least about 12 weeks (e.g., about 12 weeks), respectively, with the pharmaceutical composition. In another exemplary embodiment, the success rate, for a subject aged 35-64 years, after treatment for at least about 2 weeks (e.g., about 2 weeks), at least about 4 weeks (e.g., about 4 weeks), at least about 8 weeks (e.g., about 8 weeks), or at least about 12 weeks (e.g., about 12 weeks), is at least about 8% greater, at least about 10% greater, at least about 12% greater, at least about 15% greater, about 7% to about 60% greater, about 8% to about 60% greater, about 10% to about 60% greater, about 12% to about 60% greater, about 15% to about 60% greater, about 7% to about 50% greater, about 8% to about 50% greater, about 10% to about 50% greater, about 12% to about 50% greater, about 15% to about 50% greater, about 7% to about 40% greater, about 8% to about 40% greater, about 10% to about 40% greater, about 12% to about 40% greater, about 15% to about 40% greater, about 7% to about 30% greater, about 8% to about 30% greater, about 10% to about 30% greater, about 12% to about 30% greater, or about 15% to about 30% greater, than a success rate achieved after treatment of a subject aged 18-35 years with the pharmaceutical composition.

In other exemplary embodiments, the success rate, for subjects aged 35-64 years and after treatment for at least about 2 weeks, at least about 4 weeks, at least about 8 weeks, or at least about 12 weeks with the pharmaceutical composition, is at least about 3% greater, at least about 4% greater, at least about 5% greater, at least about 6% greater, about 3% to about 60% greater, about 3% to about 50% greater, about 3% to about 40% greater, about 3% to about 30% greater, about 4% to about 60% greater, about 4% to about 50% greater, about 4% to about 40% greater, about 4% to about 30% greater, about 5% to about 60% greater, about 5% to about 50% greater, about 5% to about 40% greater, about 5% to about 30% greater, about 6% to about 60% greater, about 6% to about 50% greater, about 6% to about 40% greater, or about 6% to about 30% greater, than a success rate achieved after treatment of subjects aged 18-35 years for at least about 2 weeks, at least about 4 weeks, at least about 8 weeks, or at least about 12 weeks, respectively, with the pharmaceutical composition.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 2 weeks (e.g., about 2 weeks), at least about 4 weeks (e.g., about 4 weeks), at least about 8 weeks (e.g., about 8 weeks), or at least about 12 weeks (e.g., about 12 weeks), to achieve, in a group of such subjects, a mean percentage decrease, from baseline, in the number of inflammatory lesions of at least about 38%, at least about 52%, at least about 61%, or at least about 62%, respectively.

In other exemplary embodiments, the mean percentage decrease in the number of inflammatory lesions after at least about 2 weeks (e.g., about 2 weeks) is about 38% to about 100%, about 38% to about 95%, about 38% to about 90%, about 38% to about 80%, about 38% to about 70%, about 38% to about 60%, about 38% to about 50%, or about 38% to about 40%.

In other exemplary embodiments, the mean percentage decrease in the number of inflammatory lesions after at least about 4 weeks (e.g., about 4 weeks) is about 52% to about 100%, about 52% to about 95%, about 52% to about 90%, about 52% to about 80%, about 52% to about 70%, or about 52% to about 60%.

In other exemplary embodiments, the mean percentage decrease in the number of inflammatory lesions after at least about 8 weeks (e.g., about 8 weeks) is about 61% to about 100%, about 61% to about 95%, about 61% to about 90%, about 61% to about 80%, or about 61% to about 70%.

In other exemplary embodiments, the mean percentage decrease in the number of inflammatory lesions after at least about 12 weeks (e.g., about 12 weeks) is about 62% to about 100%, about 62% to about 95%, about 62% to about 90%, about 62% to about 80%, or about 62% to about 70%.

In other exemplary embodiments, said pharmaceutical composition is applied (or capable of being applied) once daily for a period of at least about 2 weeks (e.g., about 2 weeks), at least about 4 weeks (e.g., about 4 weeks), at least about 8 weeks (e.g., about 8 weeks), or at least about 12 weeks (e.g., about 12 weeks), to achieve, in a group of such subjects, a mean percentage decrease, from baseline, in the number of inflammatory lesions of at least about 42%, at least about 58%, at least about 68%, or at least about 69%, respectively.

In another exemplary embodiment, the mean percentage decrease in the number of inflammatory lesions after at least about 2 weeks (e.g., about 2 weeks) is at least about 45%, at least about 50%, at least about 55%, or at least about 60%. In other exemplary embodiments, the mean percentage decrease in the number of inflammatory lesions after at least about 2 weeks (e.g., about 2 weeks) is about 42% to about 100%, about 42% to about 95%, about 42% to about 90%, about 42% to about 80%, about 42% to about 70%, about 42% to about 60%, or about 42% to about 50%. In other exemplary embodiments, the mean percentage decrease in the number of inflammatory lesions after at least about 2 weeks (e.g., about 2 weeks) is about 45% to about 100%, about 45% to about 95%, about 45% to about 90%, about 45% to about 80%, about 45% to about 70%, about 45% to about 60%, or about 45% to about 50%. In other exemplary embodiments, the mean percentage decrease in the number of inflammatory lesions after at least about 2 weeks (e.g., about 2 weeks) is about 50% to about 100%, about 50% to about 95%, about 50% to about 90%, about 50% to about 80%, about 50% to about 70%, or about 50% to about 60%. In other exemplary embodiments, the mean percentage decrease in the number of inflammatory lesions after at least about 2 weeks (e.g., about 2 weeks) is about 55% to about 100%, about 55% to about 95%, about 55% to about 90%, about 55% to about 80%, about 55% to about 70%, or about 55% to about 60%. In other exemplary embodiments, the mean percentage decrease in the number of inflammatory lesions after at least about 2 weeks (e.g., about 2 weeks) is about 60% to about 100%, about 60% to about 95%, about 60% to about 90%, about 60% to about 80%, or about 60% to about 70%.

In another exemplary embodiment, the mean percentage decrease in the number of inflammatory lesions after at least about 4 weeks (e.g., about 4 weeks) is at least about 60%, at least about 65%, at least about 70%, or at least about 75%. In other exemplary embodiments, the mean percentage decrease in the number of inflammatory lesions after at least about 4 weeks (e.g., about 4 weeks) is about 58% to about 100%, about 58% to about 95%, about 58% to about 90%, about 58% to about 80%, about 58% to about 70%, or about 58% to about 60%. In other exemplary embodiments, the mean percentage decrease in the number of inflammatory lesions after at least about 4 weeks (e.g., about 4 weeks) is about 60% to about 100%, about 60% to about 95%, about 60% to about 90%, about 60% to about 80%, or about 60% to about 70%. In other exemplary embodiments, the mean percentage decrease in the number of inflammatory lesions after at least about 4 weeks (e.g., about 4 weeks) is about 65% to about 100%, about 65% to about 95%, about 65% to about 90%, about 65% to about 80%, or about 65% to about 70%. In other exemplary embodiments, the mean percentage decrease in the number of inflammatory lesions after at least about 4 weeks (e.g., about 4 weeks) is about 70% to about 100%, about 70% to about 95%, about 70% to about 90%, or about 70% to about 80%. In other exemplary embodiments, the mean percentage decrease in the number of inflammatory lesions after at least about 4 weeks (e.g., about 4 weeks) is about 75% to about 100%, about 75% to about 95%, about 75% to about 90%, or about 75% to about 80%.

In another exemplary embodiment, the mean percentage decrease in the number of inflammatory lesions after at least about 8 weeks (e.g., about 8 weeks) is at least about 70%, at least about 75%, at least about 80%, or at least about 85%. In other exemplary embodiments, the mean percentage decrease in the number of inflammatory lesions after at least about 8 weeks (e.g., about 8 weeks) is about 68% to about 100%, about 68% to about 95%, about 68% to about 90%, about 68% to about 80%, or about 68% to about 70%. In other exemplary embodiments, the mean percentage decrease in the number of inflammatory lesions after at least about 8 weeks (e.g., about 8 weeks) is about 70% to about 100%, about 70% to about 95%, about 70% to about 90%, or about 70% to about 80%. In other exemplary embodiments, the mean percentage decrease in the number of inflammatory lesions after at least about 8 weeks (e.g., about 8 weeks) is about 75% to about 100%, about 75% to about 95%, about 75% to about 90%, or about 75% to about 80%. In other exemplary embodiments, the mean percentage decrease in the number of inflammatory lesions after at least about 8 weeks (e.g., about 8 weeks) is about 80% to about 100%, about 80% to about 95%, or about 80% to about 90%. In other exemplary embodiments, the mean percentage decrease in the number of inflammatory lesions after at least about 8 weeks (e.g., about 8 weeks) is about 85% to about 100%, about 85% to about 95%, or about 85% to about 90%.

In another exemplary embodiment, the mean percentage decrease in the number of inflammatory lesions after at least about 12 weeks (e.g., about 12 weeks) is at least about 70%, at least about 75%, at least about 80%, or at least about 85%. In other exemplary embodiments, the mean percentage decrease in the number of inflammatory lesions after at least about 12 weeks (e.g., about 12 weeks) is about 69% to about 100%, about 69% to about 95%, about 69% to about 90%, about 69% to about 80%, or about 69% to about 70%. In other exemplary embodiments, the mean percentage decrease in the number of inflammatory lesions after at least about 12 weeks (e.g., about 12 weeks) is about 70% to about 100%, about 70% to about 95%, about 70% to about 90%, or about 70% to about 80%. In other exemplary embodiments, the mean percentage decrease in the number of inflammatory lesions after at least about 12 weeks (e.g., about 12 weeks) is about 75% to about 100%, about 75% to about 95%, about 75% to about 90%, or about 75% to about 80%. In other exemplary embodiments, the mean percentage decrease in the number of inflammatory lesions after at least about 12 weeks (e.g., about 12 weeks) is about 80% to about 100%, about 80% to about 95%, or about 80% to about 90%. In other exemplary embodiments, the mean percentage decrease in the number of inflammatory lesions after at least about 12 weeks (e.g., about 12 weeks) is about 85% to about 100%, about 85% to about 95%, or about 85% to about 90%.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 2 weeks (e.g., about 2 weeks), to achieve, in a group of such subjects, a mean percentage decrease, from baseline, in the number of inflammatory lesions of at least about 42% compared to a mean percentage decrease, from baseline, in the inflammatory lesions of at least about 23% after treatment with vehicle control.

In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 23% after treatment with vehicle control.

In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 42% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is at least about 42% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 45% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 50% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 55% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 23% after treatment with vehicle control. In other exemplary embodiments, the mean percentage decrease in the number of inflammatory lesions after at least about 2 weeks (e.g., about 2 weeks) is about 40% to about 70% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 40% to about 70% compared to a mean percentage decrease in the number of inflammatory lesions of about 23% to about 25% after treatment with vehicle control.

In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 23% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of at least about 23% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 25% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 30% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 35% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 20% to about 50% after treatment with vehicle control. In other exemplary embodiments, the mean percentage decrease in the number of inflammatory lesions after application of the pharmaceutical composition for at least about 2 weeks (e.g., about 2 weeks) is about 42% to about 45% compared to a mean percentage decrease in the number of inflammatory lesions of about 23% to about 25% after treatment with vehicle control.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 4 weeks (e.g., about 4 weeks) to achieve, in a group of such subjects, a mean percentage decrease, from baseline, in the number of inflammatory lesions of at least about 58% compared to a mean percentage decrease, from baseline, in the inflammatory lesions of at least about 36% after treatment with vehicle control.

In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 36% after treatment with vehicle control.

In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 58% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is at least about 58% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 60% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 65% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 70% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 36% after treatment with vehicle control. In other exemplary embodiments, the mean percentage decrease in the number of inflammatory lesions after at least about 4 weeks (e.g., about 4 weeks) is about 55% to about 75% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 55% to about 75% compared to a mean percentage decrease in the number of inflammatory lesions of about 36% to about 38% after treatment with vehicle control.

In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 36% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of at least about 36% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 40% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 45% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 50% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 35% to about 55% after treatment with vehicle control. In other exemplary embodiments, the mean percentage decrease in the number of inflammatory lesions after application of the pharmaceutical composition for at least about 4 weeks (e.g., about 4 weeks) is about 58% to about 60% compared to a mean percentage decrease in the number of inflammatory lesions of about 36% to about 38% after treatment with vehicle control.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of at least about 8 weeks (e.g., about 8 weeks) to achieve, in a group of such subjects, a mean percentage decrease, from baseline, in the number of inflammatory lesions of at least about 68% compared to a mean percentage decrease, from baseline, in the inflammatory lesions of at least about 42% after treatment with vehicle control.

In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 42% after treatment with vehicle control.

In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 68% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is at least about 68% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 70% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 75% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 80% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 42% after treatment with vehicle control. In other exemplary embodiments, the mean percentage decrease in the number of inflammatory lesions after at least about 8 weeks (e.g., about 8 weeks) is about 65% to about 85% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 65% to about 85% compared to a mean percentage decrease in the number of inflammatory lesions of about 42% to about 44% after treatment with vehicle control.

In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 42% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of at least about 42% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 8 weeks (e.g., about 8 weeks) is about 68% to about 70% compared to a mean percentage decrease in the number of inflammatory lesions of about 42% to about 44% after treatment with vehicle control.

Another exemplary embodiment is a pharmaceutical composition for use in the treatment of rosacea in subjects aged 35-64 years, said pharmaceutical composition comprising from about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied (or capable of being applied) once daily for a period of least about 12 weeks (e.g., about 12 weeks), to achieve, in a group of such subjects, a mean percentage decrease, from baseline, in the number of inflammatory lesions of at least about 69%, compared to a mean percentage decrease, from baseline, in the inflammatory lesions of at least about 41% after treatment with vehicle control.

In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 41% after treatment with vehicle control.

In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 69% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is at least about 69% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 70% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 75% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 80% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 41% after treatment with vehicle control. In other exemplary embodiments, the mean percentage decrease in the number of inflammatory lesions after at least about 12 weeks (e.g., about 12 weeks) is about 65% to about 85% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 65% to about 85% compared to a mean percentage decrease in the number of inflammatory lesions of about 41% to about 43% after treatment with vehicle control.

In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 41% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of at least about 41% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 45% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 50% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 55% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 40% to about 60% after treatment with vehicle control. In other exemplary embodiments, the mean percentage decrease in the number of inflammatory lesions after application of the pharmaceutical composition for at least about 12 weeks (e.g., about 12 weeks) is about 69% to about 71% compared to a mean percentage decrease in the number of inflammatory lesions of about 41% to about 43% after treatment with vehicle control.

In other exemplary embodiments, the pharmaceutical composition comprises about 2.5% w/w to about 10% w/w of benzoyl peroxide, preferably about 3% to about 9%, about 4% to about 8%, and more preferably about 5% w/w of benzoyl peroxide.

In other exemplary embodiments, the benzoyl peroxide is in a form selected from the group consisting of solid, solution, or suspension.

In other exemplary embodiments, the pharmaceutical composition is applied (or capable of being applied) as a first line therapy for the treatment of rosacea. In some exemplary embodiments, the pharmaceutical composition is applied (or capable of being applied) as a first line therapy for the treatment of rosacea in a subject aged 35-64 years.

In other exemplary embodiments, the rosacea is any of erythematotelangiectatic rosacea, papulopustular rosacea, phymatous rosacea, ocular rosacea, and combinations thereof.

In other exemplary embodiments, the rosacea is moderate to severe rosacea, preferably severe rosacea.

In other exemplary embodiments, the pharmaceutical composition is a cream or an emulsion.

In other exemplary embodiments, the pharmaceutical composition is an extended release formulation.

In other exemplary embodiments, the benzoyl peroxide is present in an inert system that provides a barrier between the benzoyl peroxide and its surrounding environment.

In other exemplary embodiments, the benzoyl peroxide is released (or capable of release) in predetermined amounts over a specified period of time

In other exemplary embodiments, the extended-release effect is obtained by encapsulation, microencapsulation, microspheres, a coating, or a combination thereof, and the like.

In other exemplary embodiments, the benzoyl peroxide is encapsulated or micro encapsulated.

In other exemplary embodiments, the benzoyl peroxide is included in a microsphere or a coating.

In other exemplary embodiments, the pharmaceutical composition is applied (or capable of being applied) to the skin of a subject aged 40-64 years, 45-64 years, 50-64 years, 55-64 years, or 60-64 years, in need of said treatment. In other exemplary embodiments, the pharmaceutical composition is applied (or capable of being applied) to the skin of a subject aged 35-60 years, 35-55 years, 35-50 years, 35-45 years, or 35-40 years in need of said treatment.

In some further embodiments, the composition further comprises at least one non pharmaceutical active additive selected from the group consisting of chelating agents, antioxidants, sunscreens, preservatives, fillers, electrolytes, humectants, dyes, mineral or organic acids or bases, fragrances, essential oils, moisturizers, vitamins, essential fatty acids, sphingolipids, self-tanning compounds, calmatives and skin-protecting agents, pro-penetrating agents and gelling agents, or a mixture and/or combination thereof.

In other embodiments, the composition is formulated into a topically applicable, physiologically acceptable medium comprising of: (a) at least one member selected from the group consisting of water, alcohols, oils, fatty substances and waxes; and (b) at least one additive selected from the group consisting of chelating agents, antioxidants, sunscreens, preservatives, fillers, electrolytes, humectants, dyes, mineral acids, mineral bases, organic acids, organic bases, fragrances, essential oils, moisturizers, vitamins, essential fatty acids, sphingolipids, self-tanning compounds, calmatives, skin-protecting agents, pro-penetrating agents, gelling agents, emulsifiers, co-emulsifiers, and mixtures and/or combinations thereof.

In some embodiments, the composition is formulated as an emulsion (including an oil-in-water emulsion, a water-in-oil emulsion, multiple emulsions and microemulsions). In other embodiments, the composition is formulated as a cream.

The compositions described in exemplary embodiments herein are pharmaceutical compositions, and especially dermatological compositions, which can be in any galenical form conventionally used for topical application. By addition of a fatty or oily phase, they can also be in the form of dispersions of the lotion or serum type, emulsions of liquid or semi-liquid consistency of the milk type obtained by dispersing a fatty phase in an aqueous phase (O/W) or conversely (W/O), or suspensions or emulsions of soft, semiliquid or solid consistency of the cream, gel or ointment type, or alternatively multiple emulsions (W/O/W or O/W/O), microemulsions, microcapsules, microparticles and/or vesicular dispersions of ionic and/or nonionic type, and/or wax/aqueous phase dispersions. These compositions are formulated according to the usual methods.

In further embodiments, the composition comprises, as a single pharmaceutical active agent, benzoyl peroxide in a solid form, for topical use in the treatment of rosacea, is an oil in water emulsion comprising a polyoxylstearate and a glyceryl stearate. Various methods for the preparation of the BPO-containing compositions are described in U.S. Application Publication Nos. 2010/0016443, 2017/0281571 and 2018/0147165 and U.S. Pat. No. 9,687,465.

In some embodiments, the ratio of said polyoxylstearate to said glyceryl stearate is in the range of about 0.1:10 to about 10:0.1.

In yet further embodiments, said polyoxylstearate is selected from the group consisting of Polyoxyl-8 stearate, Polyoxyl-20 stearate, Polyoxyl-40 stearate, Polyoxyl-100 stearate and combinations and/or mixtures thereof.

In further embodiments, said glyceryl stearate is selected from the group consisting of glyceryl mono-stearate, glyceryl di-stearate and combinations and/or mixtures thereof.

In other embodiments, said polyoxylstearate in said composition is in the range of from about 0.1% w/w to about 30% w/w.

In further embodiments, the amount of said glyceryl stearate in said composition is in the range of from about 0.1% w/w to about 30% w/w.

In other embodiments, said composition further comprises at least one fatty alcohol.

In other embodiments, said at least one fatty alcohol is selected from the group consisting of octyl alcohol, 2-ethyl hexanol. nonyl alcohol, decyl alcohol, undecanol, dodecyl alcohol, tridecyl alcohol, tetradecyl alcohol, pentadecyl alcohol, cetyl alcohol, palmitoleyl alcohol, heptadecyl alcohol, cetostearyl alcohol, stearyl alcohol, isostearyl alcohol, elaidyl alcohol, oleyl alcohol, linoleyl alcohol, elaidolinolenyl alcohol, ricinoleyl alcohol, nonadecyl alcohol, arachidyl alcohol, heneicosyl alcohol. behenyl alcohol, erucyl alcohol, lignoceryl alcohol, ceryl alcohol, montanyl alcohol, cluytyl alcohol, myricyl alcohol, melissyl alcohol, geddyl alcohol, cetearyl alcohol and combinations and/or mixtures thereof.

In further embodiments, the amount of said at least one fatty alcohol in said composition is in the range of from about 0.2% w/w to about 50% w/w.

In yet other embodiments, said composition further comprises a polyacrylic acid homopolymer or copolymer.

In other embodiments, said oil in said oil in water emulsion is selected from the group consisting of paraffin oil, isopropyl myristate, caprylic/capric triglyceride, squalane, squalene, almond oil, castor oil, olive oil, jojoba oil, sunflower oil, soybean oil, grape seed oil, dimethicone, cyclomethicone and combinations and/or mixtures thereof.

In further embodiments, said oil in present in the composition in an amount in the range of from about 0.05% w/w to about 50% w/w.

In some embodiments, said water in said oil in water emulsion further comprises at least one water soluble humectant.

In other embodiments, said at least one water soluble humectant is selected from the group consisting of propylene glycol, glycerin, polyethylene glycol-X and combinations and/or mixtures thereof, where X is in the range of from about 200 to about 10,000.

In some embodiments, the composition comprises said solid BPO in a controlled and/or slowed release drug delivery system. In further embodiments, said controlled and/or slowed release drug delivery system is an encapsulation in a microcapsule, wherein said solid BPO is embedded in said microcapsule. When referring to a “controlled and/or slowed release drug delivery system” it should be understood to relate to a delivery system (which in the present application is a topical delivery system) that enables the release of the pharmaceutical active agent in predetermined amounts over a specified period. In some embodiments, said system is a core-shell system of a microcapsule and/or a porous matrix structure, such as, for example, a microsponge. The term “embedded” should be understood to encompass an inert system that provides a barrier between the pharmaceutical active agent, i.e. BPO, and its surrounding environment in the composition. In some embodiments, said agent is entrapped and/or encapsulated in said controlled release system.

In some embodiments, said core of said microcapsule comprises or consists of said solid BPO.

In some further embodiments, said microcapsules are a core shell microcapsule. The shell comprises at least one inorganic polymer. In some other embodiments, said inorganic polymer of said shell is a metal oxide or semi-metal oxide shell (layer).

In some embodiments, said microcapsule comprises a metal oxide or semi-metal oxide coating or layer (shell) and a core comprising or consisting of solid BPO.

In some embodiments, said microcapsule comprises a metal oxide or semi-metal oxide coating or layer (shell) and a core comprising solid BPO is prepared by a process comprising the steps of:

(a) contacting a solid BPO particulate matter with an ionic additive and an aqueous medium to obtain a dispersion of said particulate matter having positive charges on its surface;

(b) subjecting the particulate matter to a coating procedure comprising precipitating a metal oxide salt onto the surface of the particulate matter to form a metal oxide layer thereon thereby to obtain particulate matter coated by a metal oxide coating layer;

(c) repeating step (b) at least 4 more times: and

(d) aging said coating layer.

As used herein, the term “solid BPO particulate matter” refers to a solid BPO having solubility in water of less than about 1% w/w, typically less than about 0.5% and at times less than about 0.1% w/w at room temperature (about 20° C.). The “solid BPO particulate matter” constitutes the “core” of the particles obtained by the process. The solid BPO particulate matter, is, in some embodiments, in such a state of subdivision that it can be suspended in water, e.g., in the form of a finely-divided powder having a D₉₀ (see definition below), in some embodiments in the range of from about 0.3 to about 50 microns. Such a particulate matter can be readily suspended in an aqueous system by stirring, with or without the aid of a surfactant.

The terms “solid BPO particulate matter” and “particulate matter” will be used interchangeably.

In the present application, the terms “layer”, “coating” or “shell” and similar terms, refer to a layer of metal oxide or semi-metal oxide formed around a particle or particulate matter. The layer or coating need not always be complete or uniform and need not necessarily lead to complete coverage of the particulate matter or particle surface. It is appreciated that upon repetition of the coating steps as the coating process proceeds a more uniform coating and more complete coverage of the particulate matter is obtained.

The term “dispersion,” as used herein, in step (a) of the process refers to a solid dispersion of the particulate matter in the aqueous medium. Step (a) of the process can further comprise reducing the particle size of the particulate matter to the desired particle size, for example, by milling or homogenization.

The core (i.e., solid, BPO particulate matter) can be of any shape, for example, rod-like, plate-like, ellipsoidal, cubic, spherical shape, combinations thereof and the like.

Reference to the size of particles will be made through their D₉₀, which means that about 90% of the particles have the stated dimension or less (measured by volume). Thus, for example, for spherical particles stated to have a diameter of about 10 micrometers (“microns”), this means that the particles have a D₉₀ of about 10 microns. The D₉₀ can be measured by laser diffraction. For particles having a shape other than spheres, the D₉₀ refers to the mean average of the diameter of a plurality of particles.

In the case of cores having a spherical shape, the D₉₀ can be in the range of from about 0.3 to 90 microns, in some embodiments from about 0.3 to about 50 microns, in some other embodiments from about 1 to about 50 microns, in some further embodiments from about 5 to about 30 microns. As used herein, the phrase “D₉₀ can be in the range of from about 0.3 microns to about 90 microns” means about 90% by volume of the particles (in this case the particle's core) can be less than or equal to a value in the range of from about 0.3 microns to about 90 microns.

For generally cubic-shaped cores or cores having a shape resembling that of a cube, the mean size of a side can be in the range of from about 0.3 to about 80 microns, in some embodiments from about 0.3 to about 40 microns, in some further embodiments from about 0.8 to about 40 microns, in some further embodiments from about 4 to about 15 microns.

For rod-like shaped, ellipsoidal-shaped and plate-like shaped cores, the largest dimension (that of the longest axis) is typically in the range of from about 10 to about 100 microns, in some embodiments from about 15 to about 50 microns; and the smallest dimension is typically in the range of from about 0.5 to about 20 microns, in some further embodiments from about 2 to about 10 microns.

As used herein, unless otherwise indicated, the term “particle” refers to the metal oxide or semi-metal oxide coated particulate matter.

It is appreciated that some of the particles obtained by the process can at times be formed from two or more original particles of the solid BPO particulate and can accordingly include at times more than one core, such cores being separated from each other by a metal oxide region.

The weight of the solid BPO particulate (core material) based on the total weight of the particle can be in the range of from about 99% w/w to about 50% w/w, in some embodiments in the range of from about 97% w/w to about 50% w/w. The core material can be in a crystalline form, amorphous form, or combination thereof. The core material can be a cosmetic, pharmaceutical or an agrochemical active ingredient.

### EXEMPLARY EMBODIMENTS

BPO-containing compositions were prepared following the various preparation methods described in U.S. Application Publication Nos. 2010/0016443, 2017/0281571 and 2018/0147165 and U.S. Pat. No. 9,687,465, the contents of which are incorporated herein, by reference, in their entirety.

Description: A randomized, double-blind, multicenter, parallel group, active- and vehicle-controlled study of encapsulated 5% benzoyl peroxide cream (E-BPO) and vehicle cream was performed to assess the efficacy and safety of E-BPO compared to vehicle. Study duration was 12 weeks and included approximately 350 male and female patients afflicted with papulopustular rosacea (rated 3 or 4 on the 5-point IGA scale). Patients were at least 18 years of age.

Patients were admitted into the study after meeting all inclusion/exclusion criteria, including a clinical diagnosis of rosacea. Subjects with moderate to severe rosacea who were appropriate for systemic treatment were counseled regarding their treatment options by the Principal Investigator. At each visit, a 5-point IGA scale of rosacea and inflammatory lesion counts were performed and recorded.

Dosing: Patients were randomly organized in a 2:1 ratio to the study product or vehicle treatment group, respectively. Patients applied the study product once daily for 12 weeks on the face in a thin layer to provide even distribution.

Clinical and Safety Evaluations were performed at Screening, Baseline and Weeks 2, 4, 8 and 12.

Summary of Investigator Global Assessment (IGA):

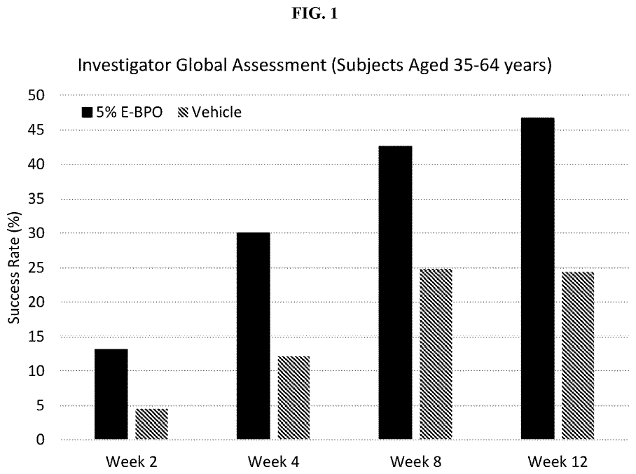

The success rate of the treatment of rosacea (defined as an IGA of Clear or Almost Clear) in patients aged 35-64 years and aged 18-35 years are shown in Table 1. The success rate of the treatment in patients aged 35-64 years is also shown in FIG. 1. For the data in Table 1, multiple imputation (MCMC) were used to impute missing values, and the percentage values for Weeks 2, 4, 8, and 12 represent average values, obtained from averaging the summary statistics generated from each imputed dataset.

The percentage of patients aged 35-64 years achieving a Clear to Almost Clear grade after application of the 5% E-BPO cream were: about 13% (week 2), about 30% (week 4), about 43% (week 8) and about 47% (week 12). The results also show that the success rate of the 5% E-BPO cream was at least about 8% (week 2), about 18% (week 4), about 18% (week 8) and about 22% (week 12) greater than the success rate achieved after treatment with vehicle alone for the corresponding amount of time.

In contrast, the percentage of patients aged 18-35 years achieving a Clear to Almost Clear grade after application of the 5% E-BPO cream was about 39% (week 12). This success rate was at least about 22% greater than the success rate achieved after treatment with vehicle alone.

As seen from these results, topical application of E-BPO is highly effective as a treatment of rosacea in patients aged 35-64 years. Up to about 47% of patients aged 35-64 achieved a clear to almost clear grade after 12 weeks of treatment with 5% E-BPO cream, which is significantly greater than the effect observed after treatment with vehicle cream or the effect observed in subjects aged 18-35 years (about 39% of patients aged 18-35 years achieved a clear to almost clear grade after 12 weeks of treatment with 5% E-BPO cream). These results show the unexpectedly superior success rate after treatment of patients aged 35-64 years with E-BPO compared to vehicle alone, or compared to treatment of patients aged 18-35 years with E-BPO.

TABLE 1

| | Percentage of Patients with Clear or Almost Clear IGA | | | | | |
|---|---|---|---|---|---|---|
| | 35-64 years | | | 18-35 years | | |
| | (5% E-BPO; | (Vehicle; | Difference | (5% E-BPO; | (Vehicle; | Difference |
| | N = 345) | N = 178) | (E-BPO − Vehicle) | N = 66) | N = 28) | (E-BPO − Vehicle) |
| Week 2 | 13.1 | 4.5 | 8.6 | | | |
| Week 4 | 30.0 | 12.1 | 17.9 | | | |
| Week 8 | 42.6 | 24.8 | 17.8 | | | |
| Week 12 | 46.7 | 24.4 | 22.3 | 39.4 | 17.9 | 21.5 |

Evaluation of Inflammatory Lesion Counts:

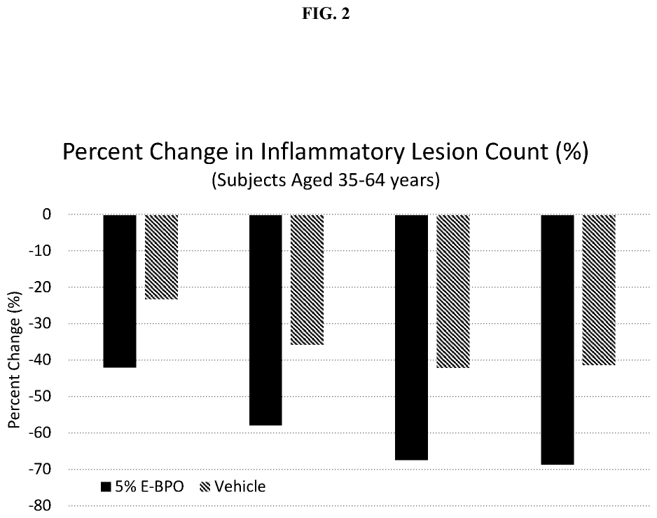

The mean percentage decrease in the number of inflammatory lesions in a group of patients aged 35-64 years after treatment with 5% E-BPO and vehicle alone, at Weeks 2, 4, 8 and 12 are shown in Table 2 and FIG. 2. The percentage values for Weeks 2, 4, 8, and 12 represent average values, obtained from averaging the summary statistics generated from each imputed dataset, and negative values represent decrease from Baseline. The mean absolute decrease in the number of inflammatory lesions are shown in Table 3. For the data in Tables 2 and 3, multiple imputation (MCMC) were used to impute missing values.

The mean percentage decrease, from Baseline, of inflammatory lesion counts in patients aged 35-64 years is: about 42% (week 2), about 58% (week 4), about 68% (week 8) and about 69% (week 12) after treatment with E-BPO compared to about 23% (week 2), about 36% (week 4), about 42% (week 8) and about 41% (week 12). The unexpectedly superior reduction in inflammatory lesion count after treatment with BPO is about 19% to about 27% higher than the reduction in inflammatory lesion counts observed after treatment with vehicle alone.

TABLE 2

| | Percent Change in Inflammatory | | |
|---|---|---|---|
| | Lesion Count from Baseline | | |
| | 35-64 years | | |
| | (5% E-BPO; | (Vehicle; | Difference |
| | N = 345) | N = 178) | (E-BPO − Vehicle) |
| Week 2 | −42.07 | −23.30 | −18.77 |
| Week 4 | −57.96 | −35.84 | −22.12 |
| Week 8 | −67.50 | −42.19 | −25.31 |
| Week 12 | −68.70 | −41.43 | −27.27 |

TABLE 3

| | Absolute Change in Inflammatory Lesion Count from Baseline | | | | | |
|---|---|---|---|---|---|---|
| | 35-64 years | | | 18-35 years | | |
| | (5% E-BPO; | (Vehicle; | Difference | (5% E-BPO; | (Vehicle; | Difference |
| | N = 345) | N = 178) | (E-BPO − Vehicle) | N = 66) | N = 28) | (E-BPO − Vehicle) |
| Week 2 | −11.8 | −6.2 | −5.6 | | | |
| Week 4 | −16.0 | −9.3 | −6.7 | | | |
| Week 8 | −18.3 | −10.9 | −7.4 | | | |
| Week 12 | −18.7 | −10.8 | −7.9 | −23.8 | −14.0 | −9.8 |

The above-discussed results demonstrate the unexpected superiority of the benzoyl peroxide composition described in this application in the treatment of rosacea in patients aged 35-64 years.

Although the exemplary embodiments of the present disclosure have been described in detail with reference to the accompanying examples and drawings, the present disclosure is not limited thereto and can be embodied in many different forms without departing from the technical concept of the present disclosure. Therefore, the exemplary embodiments of the present disclosure are provided for illustrative purposes only and are not intended to limit the technical concept of the present disclosure. The protective scope of the present disclosure should be construed based on any appended claims and combinations thereof, and all the technical concepts in the equivalent scope thereof should be construed as falling within the scope of the present disclosure. As various changes could be made in the above methods and compositions without departing from the scope of the invention, it is intended that all matter contained in the above description shall be interpreted as illustrative and not in a limiting sense. Other embodiments within the scope of the claims herein will be apparent to one skilled in the art from consideration of the specification or practice of the exemplary embodiments disclosed herein. It is intended that the specification be considered exemplary only, with the scope and spirit of the described subject matter being indicated by the claims.

What is claimed is:

1. A regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, at least about 4 weeks, at least about 8 weeks, or at least about 12 weeks, to achieve, in a group of such subjects, a success rate of at least about 12%, at least about 27%, at least about 38%, or at least about 42%, respectively, and wherein the success rate is defined as the number of subjects achieving clear or almost clear skin on the investor global assessment (IGA) scale after treatment with the pharmaceutical composition.

2. The regimen of claim 1, wherein the benzoyl peroxide is the sole active ingredient administered to the subject during the duration of the regimen.

3. The regimen of claim 2, wherein said pharmaceutical composition comprises about 5% w/w of benzoyl peroxide.

4. The regimen of claim 1, wherein the regimen is a first line therapy for the treatment of rosacea.

5. The regimen of claim 1, wherein the rosacea is any of erythematotelangiectatic rosacea, papulopustular rosacea, phymatous rosacea, ocular rosacea, and combinations thereof.

6. The regimen of claim 1, wherein the rosacea is moderate to severe rosacea.

7. The regimen of claim 1, wherein said pharmaceutical composition is a cream or an emulsion.

8. The regimen of claim 1, wherein said pharmaceutical composition is an extended-release formulation.

9. The regimen of claim 8, wherein the benzoyl peroxide is present in an inert system that provides a barrier between the benzoyl peroxide and its surrounding environment.

10. The regimen of claim 9, wherein the benzoyl peroxide is encapsulated or micro encapsulated.

11. The regimen of claim 1, wherein the success rate of said regimen is at least about 8% greater, at least about 17% greater, at least about 17% greater, or at least about 22% greater, than a success rate achieved after treatment for at least about 2 weeks, at least about 4 weeks, at least about 8 weeks, or at least about 12 weeks, respectively, with a vehicle control.

12. The regimen of claim 1, wherein the success rate of said regimen, for a subject aged 35-64 years and after treatment for at least about 12 weeks with the pharmaceutical composition, is at least about 7% greater than a success rate achieved after treatment of a subject aged 18-35 years for at least about 12 weeks with the pharmaceutical composition.

13. A regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, at least about 4 weeks, at least about 8 weeks, or at least about 12 weeks, to achieve, in a group of such subjects, a mean percentage decrease, from baseline, in the number of inflammatory lesions of at least about 38%, at least about 52%, at least about 61%, or at least about 62%, respectively.

14. The regimen of claim 13, wherein the benzoyl peroxide is the sole active ingredient administered to the subject during the duration of the regimen.

15. The regimen of claim 14, wherein said pharmaceutical composition comprises about 5% w/w of benzoyl peroxide.

16. The regimen of claim 13, wherein the regimen is a first line therapy for the treatment of rosacea.

17. The regimen of claim 13, wherein said pharmaceutical composition is a cream or an emulsion.

18. The regimen of claim 13, wherein said pharmaceutical composition is an extended-release formulation.

19. The regimen of claim 18, wherein the benzoyl peroxide is present in an inert system that provides a barrier between the benzoyl peroxide and its surrounding environment.

20. The regimen of claim 19, wherein the benzoyl peroxide is encapsulated or micro encapsulated.

21. A regimen for the therapeutic treatment of rosacea in a subject aged 35-64 years, the regimen comprising topically applying a pharmaceutical composition to the skin of the subject in need of said treatment, the pharmaceutical composition comprising about 1% w/w to about 10% w/w benzoyl peroxide as an active ingredient, and a pharmaceutically acceptable carrier or excipient, wherein the benzoyl peroxide is the only active ingredient in said pharmaceutical composition, wherein said pharmaceutical composition is applied once daily for a period of at least about 2 weeks, at least about 4 weeks, at least about 8 weeks, or at least about 12 weeks to achieve, in a group of such subjects, a mean percentage decrease, from baseline, in the number of inflammatory lesions of at least about 42%, at least about 58%, at least about 68%, or at least about 69%, respectively, compared to a mean percentage decrease, from baseline, in the inflammatory lesions of at least about 23%, at least about 36%, at least about 42%, or at least about 41%, respectively, after treatment with vehicle control.

22. The regimen of claim 21, wherein the benzoyl peroxide is the sole active ingredient administered to the subject during the duration of the regimen.

23. The regimen of claim 22, wherein the pharmaceutical composition comprises about 5% w/w of benzoyl peroxide.

24. The regimen of claim 21, wherein the regimen is a first line therapy for the treatment of rosacea.

25. The regimen of claim 21, wherein said pharmaceutical composition is a cream or an emulsion.

26. The regimen of claim 21, wherein said pharmaceutical composition is an extended-release formulation.

27. The regimen of claim 26, wherein the benzoyl peroxide is present in an inert system that provides a barrier between the benzoyl peroxide and its surrounding environment.

28. The regimen of claim 27, wherein the benzoyl peroxide is encapsulated or micro encapsulated.
